(19) United States
(12) Patent Application Publication
(10) Pub. No.: US 2011/0197293 A1
(43) Pub. Date: Aug. 11, 2011

(54) SENSE AND ANTISENSE TRANSCRIPTS OF TRPM2 AND USES THEREOF

(75) Inventors: Giovanni Lavorgna, Milano (IT); Anja Kathrin Bosserhoff, Regensburg (DE); Ann-Kathrin Wenke, Regensburg (DE)

(73) Assignees: GIOVANNI LAVORGNA, Milano (IT); UNIVERSITÄT REGENSBURG, Regensburg (DE)

(21) Appl. No.: 13/121,500

(22) PCT Filed: Sep. 23, 2009
(86) PCT No.: PCT/IB2009/054173

§ 371 Date: Apr. 21, 2011

Related U.S. Application Data
(60) Provisional application No. 61/101,123, filed on Sep. 29, 2008.

(51) Int. Cl. A01K 67/00; C07H 21/04; C12N 15/63; C12N 5/10; C12N 5/09

(52) U.S. Cl. 800/13; 536/24.5; 435/320.1; 435/325; 435/375

(57) ABSTRACT

The present invention relates to a nucleic acid molecule being able to modulate the expression of the transient receptor potential cation channel, subfamily M, member 2 (TRPM2) gene and to modulate apoptosis and uses thereof.

### FIELD OF INVENTION

The present invention relates to the identification of two tumor enriched transcripts and their shared CpG island located within the body of human TRPM2 gene on chromosome 21. The first of these transcripts is named TRPM2-AS (transient receptor potential cation channel, subfamily M, member 2-antisense). The second transcript, located across the shared CpG island, is named TRPM2-TE (tumor-enriched transient receptor potential cation channel, subfamily M, member 2). Such transcripts and their shared CpG island are useful as diagnostic, prognostic and therapeutic agents in cancer and in other diseases in which cell death by oxidative stress plays a relevant role such as neurodegenerative diseases, diabetes and stroke.

### STATE OF THE ART

There is overwhelming evidence indicating that human cancer is a genetic disease caused by sequential accumulation of mutations in oncogenes and tumor suppressor genes [1-3]. However, it is also increasingly apparent that cancer not only depends on genetic alterations, but also on epigenetic changes that collaborate with genetic traits to drive progressive stages of tumor evolution [4-6]. One of the main epigenetic cancer features is an altered DNA methylation pattern, composed of global demethylation and promoter-localized hypermethylation. While the latter phenomenon is more understood, a certain number of speculative hypotheses have been formulated about the former. For example, global hypomethylation of cancer cells could result in a de-repression of short interspersed repeated sequences (SINEs), long interspersed repeated sequences (LINEs) and retroviral sequences [7,8]. Thereby, aberrant antisense RNA transcripts would be yielded, some of which could interfere with key tumor suppressors [7]. This would then inhibit their expression and promote the evolution of malignant clones. Indeed, it would be interesting to systematically identify antisense transcripts whose expression is enriched in cancer cells, in order to study their possible role in cancerogenesis.

Over the past few years, the authors of the present invention have developed a software program called AntiHunter, aimed at identifying antisense transcripts in the Expressed Sequence Tag (EST) database [9,10]. The authors were able to apply AntiHunter to the genome-wide identification of antisense transcripts in melanoma. Several antisense transcripts showing enrichment in melanoma were identified by the program. Here, the authors report one of them, TRPM2-AS, mapped within the locus of TRPM2, an ion channel described as a member of the transient receptor potential (TRP) superfamily [11], which is a diverse group of voltage-independent calcium-permeable cation channels [12]. TRPM2 is activated by several second messengers [13-15], oxidative stress and TNF-α and is capable of mediating susceptibility to cell death [16, 17, 15, 18-20]. Moreover, a specific role for TRPM2 in cancer cell death has been recently proposed [21].

Intriguingly, graphical viewing of the AntiHunter results in a genomic browser indicated the presence of another tumor-enriched TRPM2 transcript in the same region, TRPM2-TE, located across a CpG island shared with TRPM2-AS. CpG islands are genomic regions that contain a high frequency of CG dinucleotides. In mammalian genomes, CpG islands are typically 300-3,000 base pairs in length. The “p” in CpG notation refers to the phosphodiester bond between the cytidine and the guanosine. Expression analysis of TRPM2-AS and TRPM2-TE in malignant melanoma indicated that they were consistently up-regulated. It was also determined that TRPM2-TE could contribute to the down-regulation of TRPM2 function in cancer cells and that increased expression of wild type TRPM2 in melanoma cells lead to increased susceptibility to cell death. Finally, expression analysis in other cancer types indicated that TRPM2 silencing in cancer might have an even wider role than anticipated, reinforcing the relevance of TRPM2-AS and TRPM2-TE transcripts and the shared CpG island as diagnostic, prognostic and therapeutic tools in cancer and in other diseases in which cell death by oxidative stress plays a relevant role such as neurodegenerative diseases, diabetes and stroke.

### SUMMARY OF INVENTION

In the present invention, the authors describe the computational identification of a melanoma-enriched antisense transcript, TRPM2-AS (accession number GenBank EU362988), mapped within the locus of TRPM2, an ion channel capable of mediating susceptibility to cell death. Analysis of the TRPM2-AS genomic region indicated its presence in the same region of another tumor-enriched TRPM2 transcript, TRPM2-TE (accession number GenBank EU362987), located across a CpG island shared with TRPM2-AS. Quantitative PCR experiments confirmed that TRPM2-AS and TRPM2-TE transcripts were upregulated in melanoma, and their activation was consistent with the methylation status of the shared CpG island. Functional knock-out of TRPM2-AS and TRPM2-TE, as well as over-expression of wild-type TRPM2, increased melanoma susceptibility to apoptosis and necrosis. Finally, further expression analysis in other cancer types indicated that TRPM2-AS and TRPM2-TE over-expression might have an even wider role than anticipated, reinforcing the relevance of the authors' computational approach to identifying new diagnostic/prognostic tools and therapeutic targets.

It is therefore an object of the present invention a nucleic acid molecule being able to modulate the expression of the transient receptor potential cation channel, subfamily M, member 2 (TRPM2) gene and to modulate apoptosis.

In one aspect of the invention the nucleic acid molecule is an antisense in respect to the TRPM2 gene. Preferably it has substantially the sequence of SEQ ID No. 1.

In another aspect the nucleic acid molecule is sense in respect to the TRPM2 gene. Preferably it has substantially the sequence of SEQ ID No. 2.

It is within the scope of the invention an expression vector comprising the nucleic acid molecule as above described.

It is within the scope of the invention a host cell transformed with the expression vector as above described.

It is within the scope of the invention a non human transgenic animal bearing the nucleic acid molecule as above described.

It is within the scope of the invention the nucleic acid molecule as above described for medical use, preferably for use as anti-neurodegenerative disease, more preferably if neurodegenerative disease is selected from the group of Alzheimer's or Parkinson's disease.

It is within the scope of the invention the nucleic acid molecule as above described for use as anti-apoptotic agent in ischemic cells. Preferably ischemic cells are selected from the group of: neurons, cardiomyocites, kidney cells, lung cells, pancreas beta-cells.

It is within the scope of the invention the nucleic acid molecule as above described for use as CpG island methylation agent. Preferably the nucleic acid molecule belongs to the group of methylated oligonucleotides MO1, MO2 and MO3, for use as de-novo methylation agents of the CpG island located between TRPM2-AS and TRPM2-TE transcripts.

It is within the scope of the invention the nucleic acid molecule as above described for the diagnosis and prognosis of cancer.

The nucleic acid molecule, or the vector or of the host cell are advantageously used to modulate the expression of the transient receptor potential cation channel, subfamily M, member 2 (TRPM2) gene in vivo or in vitro.

It is within the scope of the invention a molecule able to down regulate the nucleic acid molecule as above described, for use as a pro-apoptotic and/or pro-necrosis therapy agent in cancer cells.

It is within the scope of the invention a method to down-regulate TRPM2-TE and/or TRPM2-AS transcripts in melanoma cells characterized by methylating the CpG island located between TRPM2-AS and TRPM2-TE transcripts.

It is within the scope of the invention a method to induce apoptosis in melanoma cells characterized by down regulating TRPM2-TE and/or TRPM2-AS transcripts in said melanoma cells.

The invention will be now described by non limiting examples referring to the following figures:

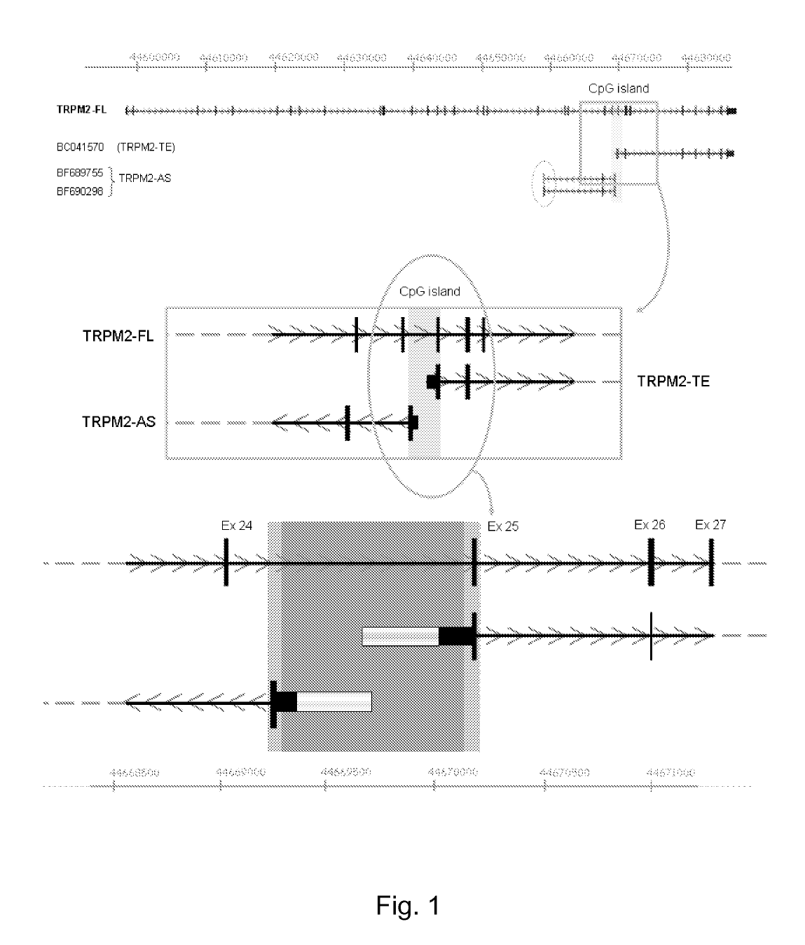

FIG. 1. Identification of an antisense transcript and related molecules within the human TRPM2 gene on chromosome 21. (Top) A genome wide search for melanoma antisense transcripts identified two antisense ESTs, BF689755 and BF690298 (circled), within the locus of TRPM2. The resulting antisense transcript mapped closely to an inner CpG island, predicted by the CpGplot program and shown in light grey, and nearby five sense EST from prostate carcinoma (only the representative EST BC041570 is shown), whose transcription seemed to originate on the other strand and in proximity to the antisense transcript. (Bottom) Primer extension experiments mapped the TRPM2-AS 5′-end in an overlapping position to the 5′-end of TRPM2-TE within intron 24 of full length TRPM2 (TRPM2-FL). The primer-extended regions of TRPM2-AS and TRPM2-TE are shown as open bars. Sequence analysis of TRPM2-TE indicated the partial ablation of exon 26 and the removal of exon 27. The predicted Pol II promoter by the FirstExon program is shown in dark grey within the mapped CpG island. Sequence coordinates for TRPM2-AS and TRPM2-TE are: 44,658,901-44,669,874 and 44,669,840-44,687,392 respectively. Numbering refers to the chromosomal position, whereas vertical bars indicate exons and arrows indicate the direction of transcription.

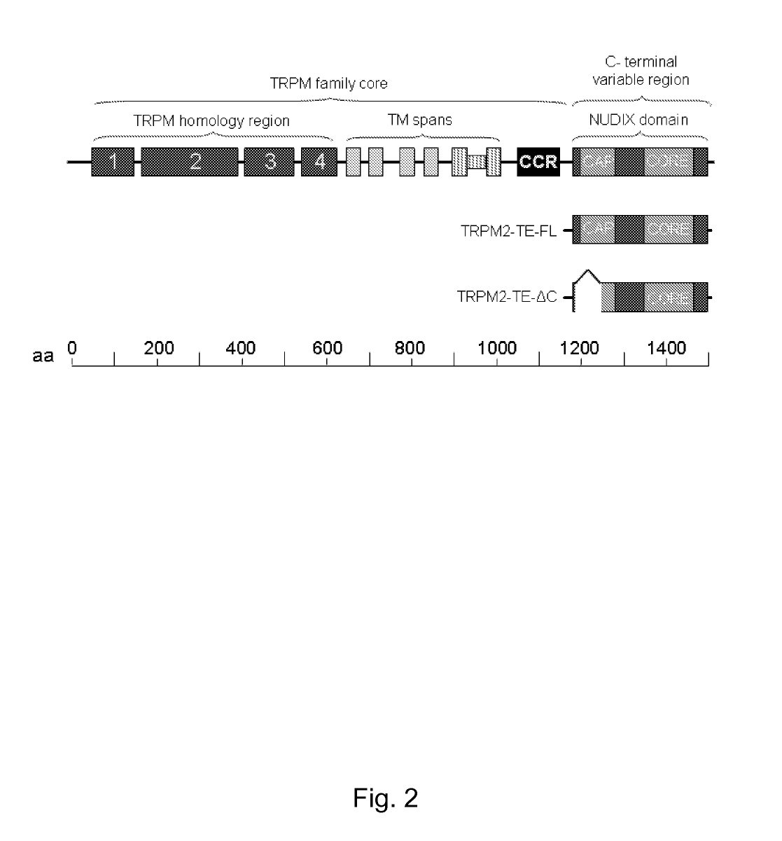

FIG. 2. Schematic representation of the TRPM2-FL, TRPM2-TE-FL and TRPM2-TE-ΔC protein products. The ‘TRPM homology’ domain and the ‘TM’ region indicate the region of highest similarity with other members of the TRPM family and the hydrophobic transmembrane span. The CORE and CAP regions within the NUDIX domain indicate the structure providing ADPRase activity and the enhancer of the CORE domain's affinity for ADPR, respectively. CCR: coiled coil region. The amino-acid scale is shown on the bottom of the figure.

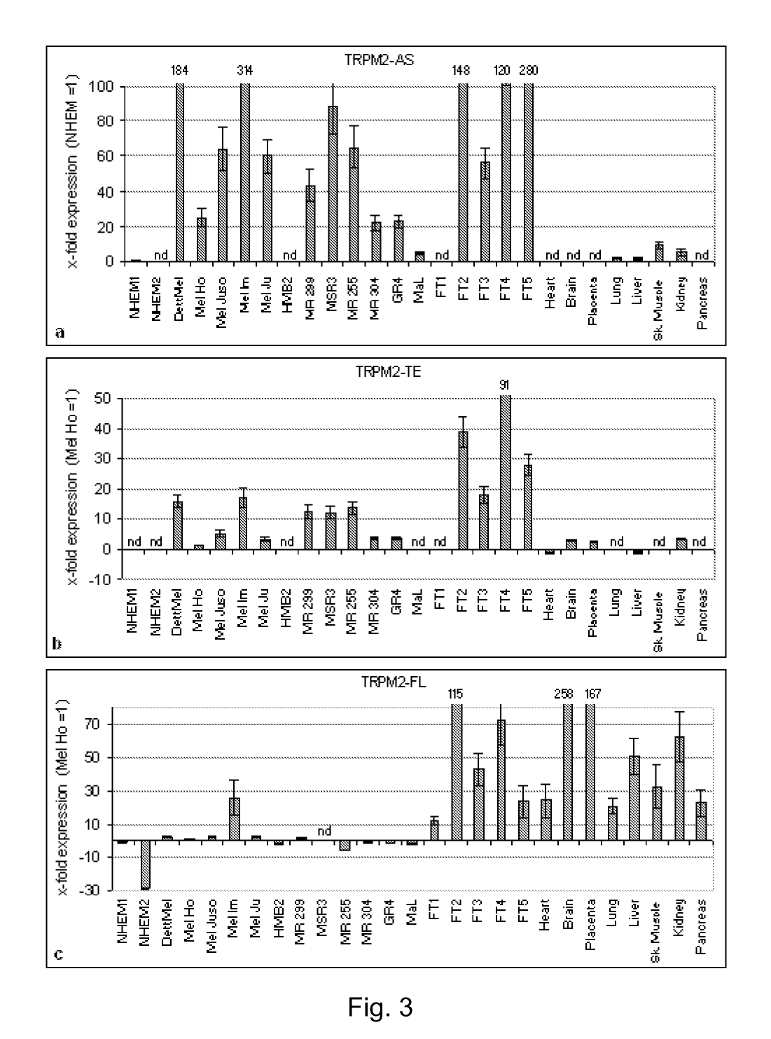

FIG. 3. Expression analysis of TRPM2-AS, TRPM2-TE and TRPM2-FL transcripts in melanoma. Quantitative PCR experiments were performed to measure the relative amounts of TRPM2-AS (a), TRPM2-TE (b) and TRPM2-FL (c) transcripts in the following set of cDNA samples: two controls from normal human melanocytes, NHEM1 and NHEM2, two cell lines derived from a primary cutaneous melanoma, Mel Ho and Mel Juso, 10 cell lines derived from metastatic melanoma, HMB2, Mel Ju, Mel Im, DettMel, GR4, MaL, MR255, MR299, MR304, MSR3, five metastatic melanoma fresh tumors, FT1, FT2, FT3, FT4, FT5, eight normal tissues: heart, brain, placenta, lung, liver, skeletal muscle, kidney, and pancreas. MaL cell line is the immortalized counterpart of the sample FT3. nd, not detected. Numbers on top of vertical bars indicate the actual off-scale value. Samples in ‘a’ were normalized against NHEM1, whereas in ‘b’ and ‘c’ they were normalized against Mel Ho. The value of the normalizer sample was equated to 1 in all panels.

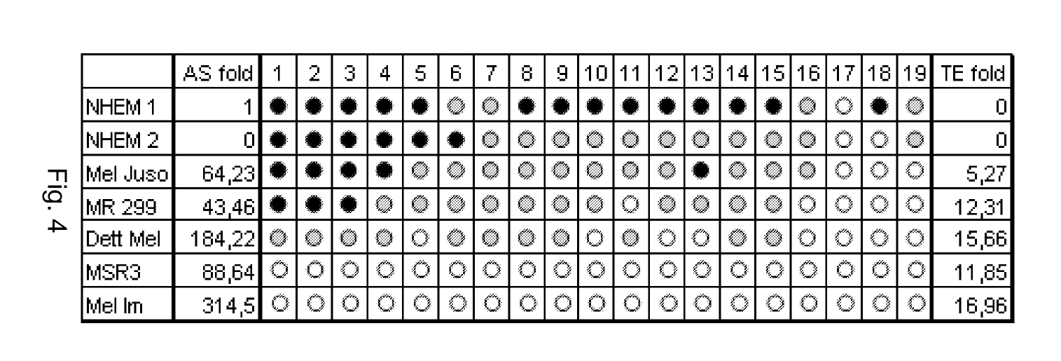

FIG. 4. CpG methylation status of the inner TRPM2 CpG island in DNA from normal and tumor cells. DNAs from melanocytes (NHEM1 and NHEM2) and melanoma cell lines (Mel Juso, MR299, DettMel, MSR3 and Mel Im) were treated with sodium bisulphite and subjected to sequencing of the TRPM2 inner CpG island. At least eight clones were sequenced for each sample. Open circles (∘), gray circles ( [symbol] ) and closed circles (), indicate methylation in 0-29%, 30-79%, 80-100% of cases respectively. The ‘AS fold’ and ‘TE fold’ columns represent the fold induction of TRPM2-AS and TRPM2-TE transcripts from FIG. 2a and FIG. 2b.

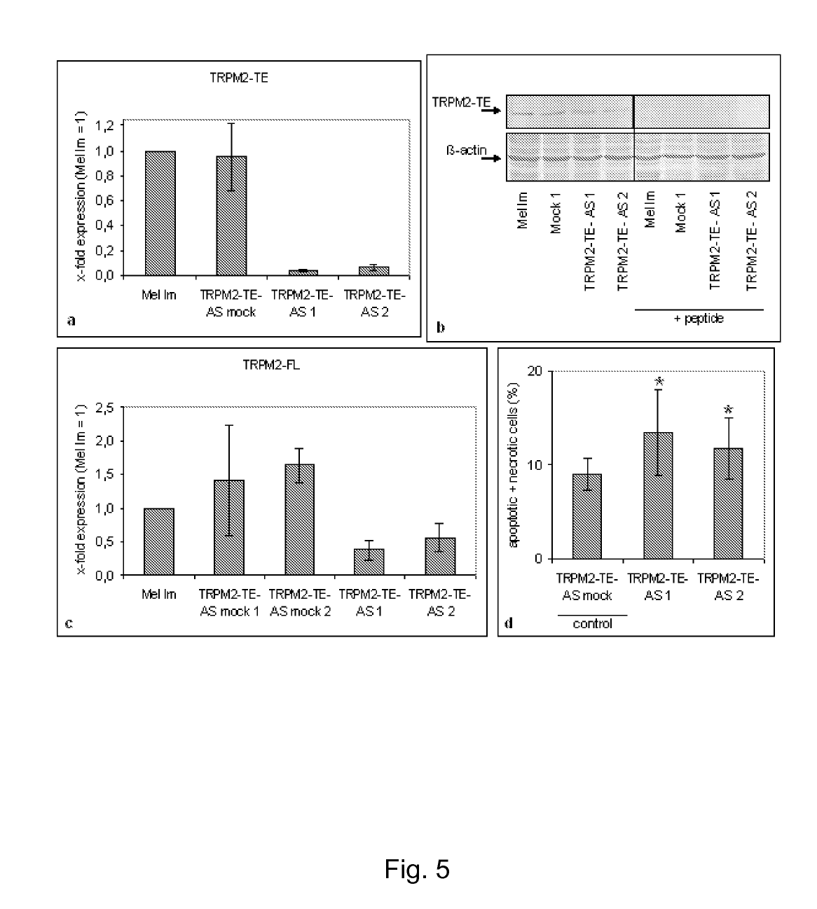

FIG. 5. Down-regulation of TRPM2-TE expression in melanoma cell line Mel Im by stable transfection with a TRPM2-TE antisense expression construct. Successful down-regulation of TRPM2-TE in cell clones 1 and 2 (TRPM2-TE-AS 1, TRPM2-TE-AS 2 was shown by quantitative RT-PCR (a) and Western blot (b). In Western blot analysis, the TRPM2-TE band was specifically competed by the peptide used for raising the antibody, but was not competed by an unrelated peptide (data not shown). β-actin was used as a loading control. TRPM2-FL expression was partially reduced by the overexpression of the TRPM2-TE antisense construct. (c). The amount of apoptosis was measured in cell clones with down-regulated TRPM2-TE expression and in control cells. Without stimulation, TRPM2-TE-AS cell clones showed higher levels of apoptotic and necrotic cells compared to mock control cells. Stimulation with H₂O₂ enhanced this effect (data not shown) (d).*, p<0.05.

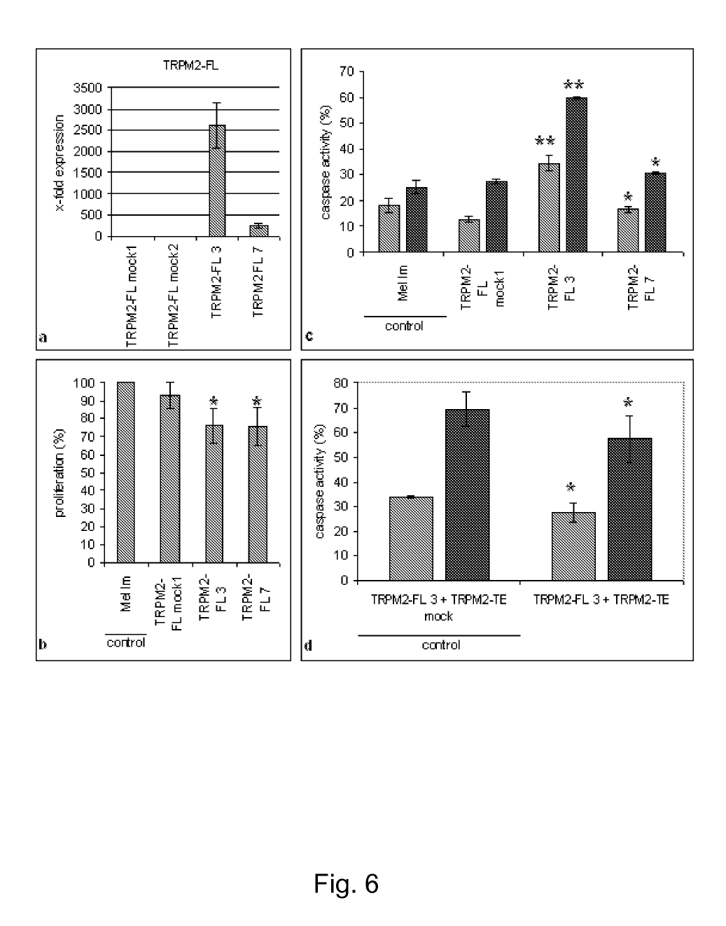

FIG. 6. Analysis of melanoma cell clones with high expression of TRPM2-FL. Stable transfection of Mel Im cells with an expression construct for TRPM2-FL was confirmed by quantitative RT-PCR showing the successful overexpression of TRPM2-FL in cell clones 3 and 7 compared to the mock transfected cell clones mock 1 and mock 2 (a). TRPM2-FL cell clones, expressing high levels of TRPM2-FL, showed a longer doubling time in proliferation assays compared to mock transfected cells and Mel Im cells (b). Measurement of apoptotic cells using a caspase assay showed that TRPM2-FL expressing cell clones had higher levels of caspase activity compared to Mel Im cells and mock transfected cell clones. This effect could be intensified by stimulating cells with H₂O₂ (dark bars) (c). Transient transfection of TRPM2-FL cell clone 3 with an expression plasmid for TRPM2-TE, protected these cells from apoptosis. This effect was even stronger after stimulating cells with H₂O₂ (dark bars) (d) *, p<0.05; **, p<0.01.

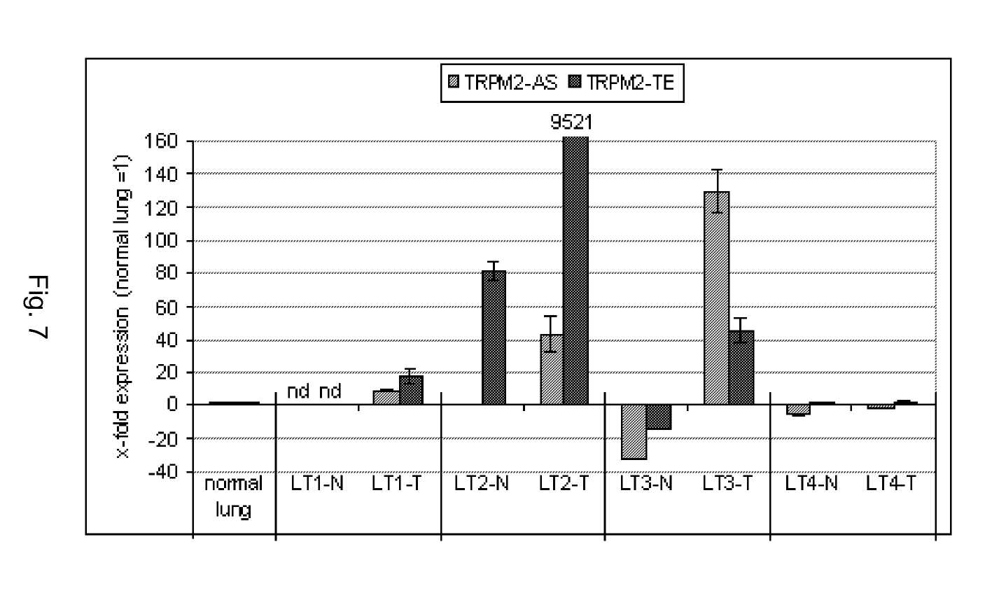

FIG. 7. TRPM2-AS and TRPM2-TE are overexpressed in matched neoplastic/non-neoplastic samples of lung biopsies. Quantitative PCR experiments were performed to measure the relative amounts of TRPM2-AS (grey) and TRPM2-TE (dark) in four matched neoplastic (samples labelled T)/non-neoplastic (samples labelled N) lung tissue (LT) samples, LT1_N and LT1_T, LT2_N and LT2_T, LT3_N and LT3_T, LT4_N and LT4_T. Results are shown as fold induction with respect to a lung cDNA from an independent healthy donor. nd, not detected.

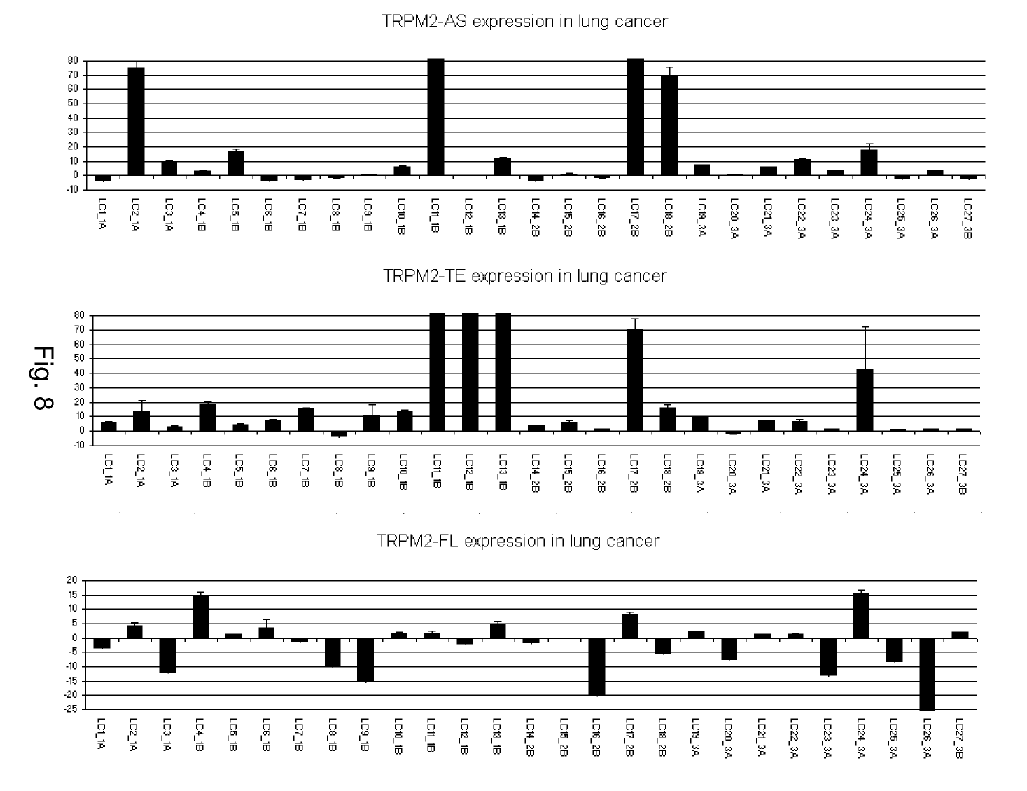

FIG. 8. Expression analysis of TRPM2-AS, TRPM2-TE and TRPM2-FL transcripts in lung cancer. Quantitative PCR experiments were performed to measure the relative amounts of TRPM2-AS (A), TRPM2-TE (B) and TRPM2-FL (C) transcripts in one cDNA from normal lung and in 27 samples from lung cancer. All samples are normalized against cDNA from normal lung. The value of the normalizer sample was equated to 1 in all panels. n.d.: not detected.

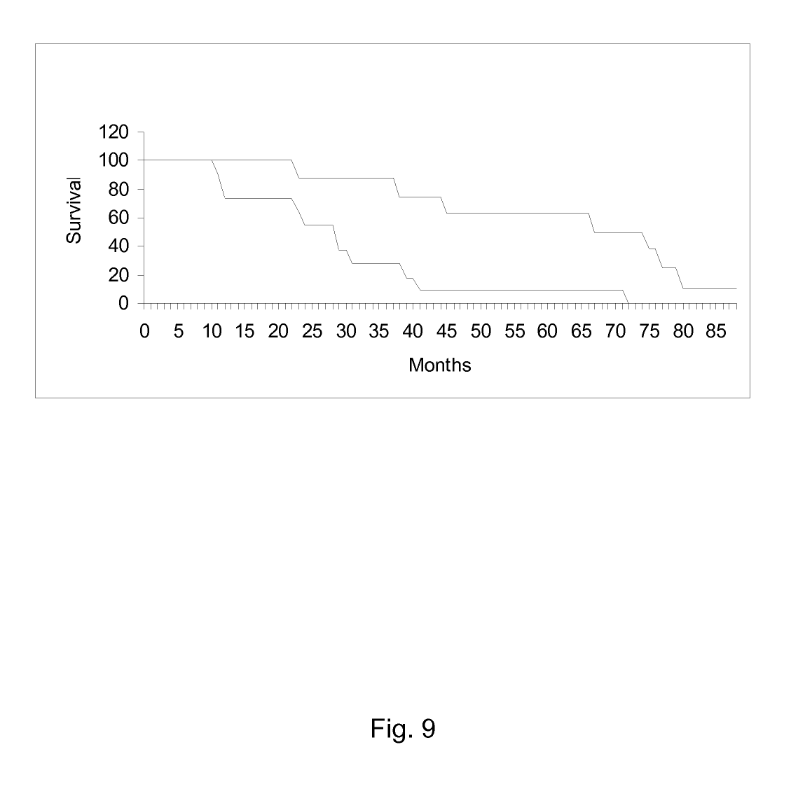

FIG. 9. Kaplan-Meier plot survival of patients from FIG. 8. The month survival of patients from stage I and II having a TRFI<−10 (dotted line; n=11; average survival: 29 months) and a TRFI>−10 (solid line; n=7; average survival: 67 months) was plotted. A log rank test determined the significance of these different distributions (p-value: 0.001).

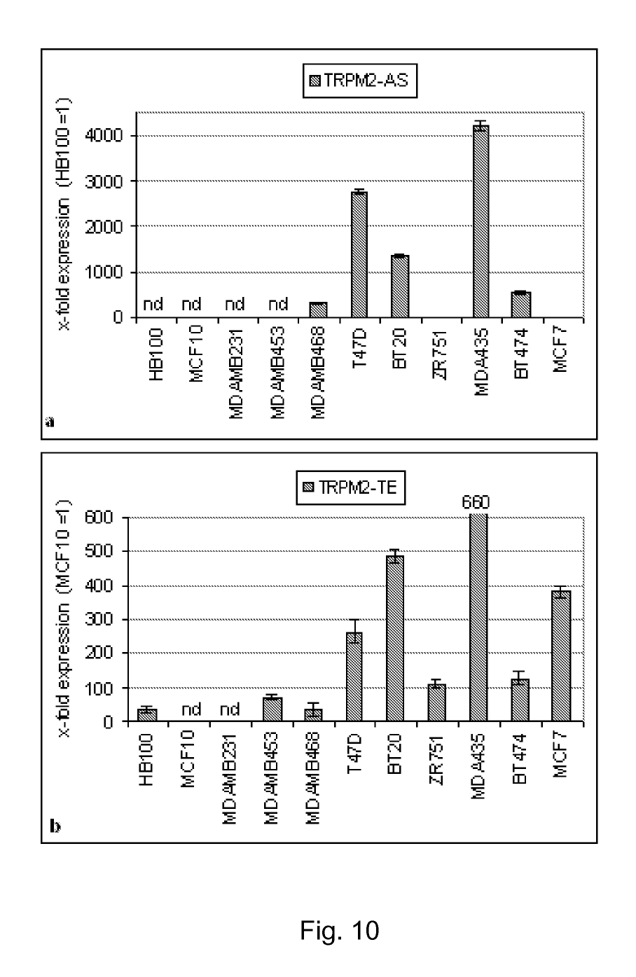

FIG. 10. TRPM2-AS and TRPM2-TE are overexpressed in several breast cancer cell lines. Quantitative RT-PCR experiments were performed to determine the relative amount of TRPM2-AS (a) and TRPM2-TE (b) transcripts in normal (HB100, MCF-10) and breast cancer cell lines (MDA-MB-231, MDA-MB-453, MDA-MB-468, T-47D, BT-20, ZR-75-1, MDA-435, BT-474, MCF-7). Results are shown as arbitrary units. nd, not detected.

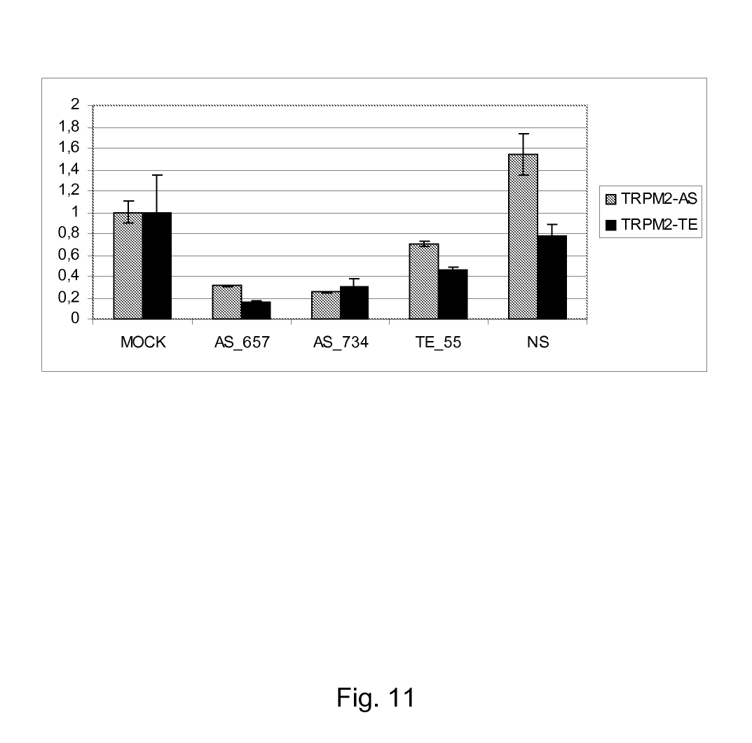

FIG. 11. qPCR results obtained after siRNA transfections in MelIm a cells. Two siRNAs designed to target the TRPM2-AS transcript (TRPM2-AS_{—}657 and TRPM2-AS_{—}734), one siRNA designed to target the TRPM2-TE transcript (TRPM2-TE 55) and a control (non-specific) siRNA were synthesized by MWG biotech (Germany, see method). TRPM2-AS_{—}657 knocks-out 69% of TRPM2-AS transcripts and 84% of TRPM2-TE molecules in Mel Im cells. TRPM2-AS_{—}734 knocks-out 75% of TRPM2-AS transcripts and 70% of TRPM2-TE molecules in Mel Im cells. TRPM2-TE 55 knocks-out 56% of TRPM2-TE transcripts and 30% of TRPM2-AS molecules in Mel Im cells.

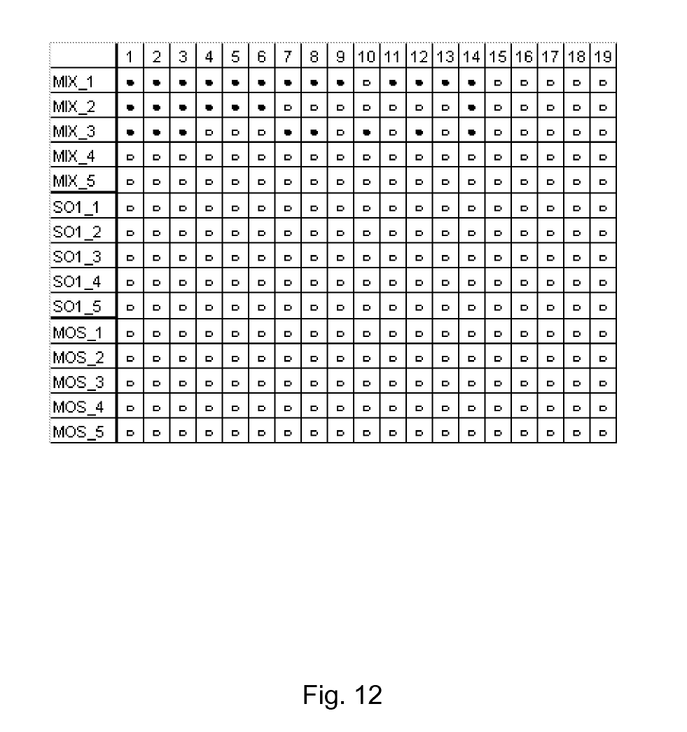

FIG. 12. CpG methylation status of the inner TRPM2 CpG island in DNA from the melanoma Mel Im cell line. This cell line was transfected with either a) a mixture of three methylated oligonucleotides, MO1, MO2 and M3, complementary to regions of the CpG island (MIX) or b) a control oligonucleotide, S01, identical at MO1 except that it doesn't contain m⁵C residues as MO1 (SO1) or c) a random methylated oligonucleotide, MOS, not significantly related to the any sequence in the human genome. DNA was extracted, treated with sodium bisulphite and subject to sequencing of the inner TRPM2 CpG island. Five clones were sequenced for each transfection. Open (∘) and closed () circles indicate, respectively, absence or presence of methylation at the CpG sites.

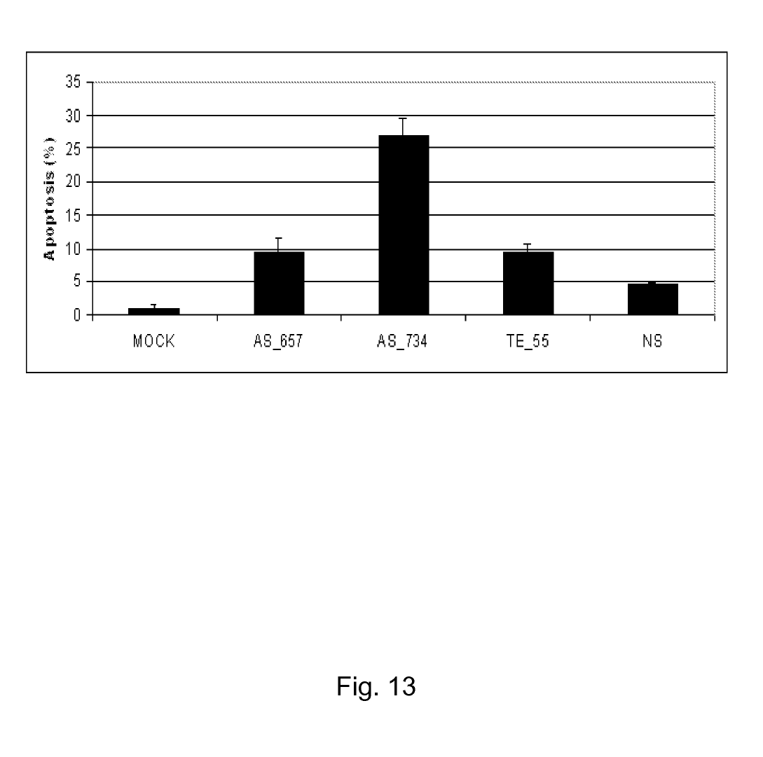

FIG. 13. Cell death is induced by knocking down TRPM2-AS or TRPM2-TE transcripts with a siRNA. Melanoma cells were transfected with the same siRNAs targeting TRPM2-AS or TRPM2-TE expression described in FIG. 11, namely TRPM2-AS_{—}657 and TRPM2-AS_{—}734 (TRPM2-AS) and TRPM2-TE_{—}55 (TRPM2-TE). Apoptosis was measured 72 h after transfection by flow cytometry. As a result, all three experimental siRNAs showed a significant increase in the level of apoptosis vs. the control siRNA or the mock transfected cells.

### DETAILED DESCRIPTION OF THE INVENTION

### Materials and Methods

Computational identification of antisense transcripts in melanoma. The AntiHunter software [9,10], capable of performing genome wide searches for antisense transcripts, was used for the identification of antisense ESTs from melanoma. Antisense ESTs identified by the program were visualized in their genomic context using the UCSC genome browser (http://genome.ucsc.edu/), in order to prioritize for further analysis those showing expression enriched in cancer tissues.

### Cells and Cell Culture.

The melanoma cell lines Mel Ho, Mel Juso, Mel Ju, Mel Im and HMB2 were described previously [36], whereas the cell lines DettMel, GR4, MaL, MR255, MR299, MR304 and MSR3 were from Hospital San Raffaele melanoma patient's repository. The cell lines Mel Ho and Mel Juso were derived from primary cutaneous melanomas; Mel Im, Mel Ju, HMB2, DettMel, GR4, MaL, MR255, MR299, MR304 and MSR3 were derived from metastases of malignant melanomas. Cells and normal human epidermal melanocytes (NHEM) were cultured as previously described [37].

### Isolation of Tumorous and Non-Tumorous Human Tissues.

Tissue samples from primary human melanoma and melanoma metastasis obtained from patients undergoing surgical treatment were immediately snap frozen and stored at −80° C. Informed consent was obtained from all patients and investigations were according to institutional guidelines and to the Declaration of Helsinki principles.

### RNA Analysis.

Confluent cells (1-5×10⁶) were trypsinised, pelleted and RNA extracted with RNeasy Mini Kit (Qiagen) according to the manufacturer's instructions. On column DNase digestion was performed with 80 μl of DNaseI (Invitrogen) for 40 minutes at room temperature. The eluted RNAs were quantified with a Nanodrop while their integrity was controlled on a 1% agarose gel. Subsequently cDNAs were generated by a reverse-transcriptase reaction performed using the ThermoScript RT-PCR System (Invitrogen). Briefly, 1 μg of total cellular RNA, 1 μl of dNTPs (10 mmol/L) and 1 μl of dN6-primer (50 ng/μl), were mixed and incubated for 5 minutes at 65° C. Then, 1 μl of Thermoscript reverse transcriptase, 4 μl of 5× first strand buffer, 1 μl of 0.1 mol/L DTT, 1 μl of RNaseOUT (40 U/μl), and sterile RNase-free water, were added to a 20 μl total reaction volume. In order to estimate the amount of genomic DNA contamination in our preparations, each RNA was used to produce a parallel RT-minus reaction where the RT-enzyme was substituted with water. Reactions were incubated for 10 minutes at 25° C., and the RNAs were then transcribed for 1 hour at 55° C. Subsequently, reverse transcriptase was inactivated at 85° C. for 5 minutes and RNA was degraded by digestion with 1 μl of RNase H (2 U/μl) at 37° C. for 20 minutes. cDNAs were controlled by PCR amplification of Beta-actin.

5′- and 3′-Rapid Amplification of cDNA Ends (RACE).

Human XG Malignant Melanoma (A375) marathon-ready cDNA (Clontech, Mountain View, Calif.) was used as template for PCR according to the manufacturer's instructions. Primer sequences are available on request. The largest-size PCR products were gel purified by the Illustra™ DNA and gel band purification kit (GE Healthcare, Buckinghamshire, UK) and cloned into a pGEM®-T Easy Vector System (Promega). Individual clones were sequenced using the DYEnamic ET Dye Terminator kit (Amersham Biosciences).

### Western Blot Analysis.

Aliquots of 3×10⁶ cells were lysed in 200 μl RIPA-buffer (Roche) and incubated for 15 min at 4° C. Insoluble fragments were removed by centrifugation at 13,000 rpm for 10 minutes and the supernatant lysate was stored at −20° C. For western blotting, 40 μg protein lysates were loaded and separated on 12.75% SDS-PAGE gels and subsequently blotted onto a PVDF membrane (Biorad, Hercules, Calif., USA). After blocking for 1 hour with 3% BSA/TBST (0.05% Tween) the membrane was incubated for 16 hours at 4° C. with the primary antibodies [polyclonal anti-TRPM2 antibody generated by Biogenes (Berlin, Germany), 1:2,000] and anti-beta-actin (Sigma, Deisenhofen, Germany, 1:2,500)). The peptide sequence recognized by the TRPM2 antibody reads as follows: MEVYKGYMDDPRNT (SEQ ID No. 24). Subsequently, the membrane was washed three times in TBST, incubated for 1 hour with alkaline phosphate-coupled secondary antibody (Chemicon; 1:2,000) and then washed again. Finally, immunoreactions were visualized by NBT/BCIP (Sigma) staining

### DNA Constructs, Transfections of Target Cells and Functional Assays.

A panel of Mel Im cell clones with reduced TRPM2-TE expression were established by stable transfection with an antisense expression plasmid (base −312 to +18 relative to the ATG codon cloned in antisense orientation into pCMX-PL1). Mel Im cell clones with induced TRPM2-FL expression were generated by stable transfection with a sense expression plasmid containing the coding sequence of TRPM2-FL from the translation start to the stop codon. Plasmids were cotransfected with pcDNA3 (Invitrogen), containing the selectable marker for neomycin resistance. Controls received pcDNA3 alone. Transfections were performed using Lipofectamin plus (Invitrogen) according to the manufacturer's instructions. One day after transfection, cells were placed in selection medium containing 50 μg/ml G418 (Sigma). After 25 days of selection, individual G418-resistant colonies were subcloned. TRPM2-FL expression levels of these clones were checked in western blot and quantitative real-time PCR. Cell proliferation was determined using the XTT assay (Roche, Mannheim, Germany). Apoptotic cells were detected by staining with AnnexinV-FITS and propidium iodide using the AnnexinV Kit (Caltag Laboratories/Invitrogen, Karlsruhe, Germany) according to the manufacturer's manual, stained cells were measured by flow cytometry with the BD FACS Calibur System (BD Biosciences, San Jose, Calif., USA). Data were analyzed and histograms generated using the Cellquest™ software (BD Biosciences). Measurement of caspase activity was another assay used to detect rates of apoptosis. Therefore cells were stained with CaspACE™ FITC-VAD-FMK In Situ Marker (Promega, USA). 5 μM of CaspACE™ FITC-VAD-FMK In Situ Marker were added to the cell medium and cells were incubated for 20 minutes protected from light. After incubation, cells were washed with PBS twice and cell pellets were resuspended in 400 μl PBS before analyzing by flow cytometry. To enhance apoptosis, 2×10⁴ cells were seeded into each well of a six-well plate and stimulated with 2 mM H₂O₂ for 30 minutes before staining. To analyze the influence of the TRPM2-TE iso form on apoptosis, the TRPM2-FL expressing cell clones were transiently transfected with an expression plasmid for TRPM2-TE. For transient transfections, 2×10⁴ cells were seeded into each well of a six-well plate and transfected with 0.5 μg TRPM2-TE plasmid DNA using the lipofectamine plus method (Invitrogen) according to the manufacturer instructions. 24 h after transfection, cells were stimulated with 2 mM H₂O₂ for 30 minutes and caspase activity was measured as described above. All experiments were performed at least three times.

### Analysis of DNA Methylation.

Genomic DNA was isolated and purified with TRIzol reagent (Invitrogen) and quantified with a Nanodrop. Average 100 ng of each DNA sample were subjected to bisulphite modification with the MethylEasy Kit (Human Genetic Signatures) allowing the sulphonation reaction to proceed for no less than 8 hours at 55° C. Following conversion, 3 μl of the modified DNAs were PCR amplified with the primers CpG-8U 5′-TTGATTTGATTTGGTTTTTGGA-3′ (SEQ ID No. 3) and CpG-8L 5′-CAAAACAAAAACTTCCTCTATA-3′ (SEQ ID No. 4) (1 ng/μl each) in the presence of buffer 10× (Qiagen), dNTPs (2 mmol/L), 5× Solution Q (Qiagen), Taq DNA Polymerase (3U), with the reaction conditions being: 1 minute of pre-incubation at 95° C., then amplification reactions were performed by 35 repetitive cycles of denaturing for 1 minute at 95° C., annealing for 2 minutes at 55° C. and extension for 2 minutes at 72° C. A semi-nested PCR reaction was performed in same conditions except for the forward primer changed to CpG-1U 5′-TGTAGATTGTGTTTGAATTTGGTTA-3′ (SEQ ID No. 5), using 4 μl of the previous 472 bp per product as template. The resulting 370 bp product was then gel-purified, cloned and sequenced using standard procedures.

### Real Time Quantification.

Beta-actin (Exons 5-6) was amplified from cDNAs using the specific primers: LC+hBactin-U 5′-TCCTCCTGGAGAAGAGCTA-3′ (SEQ ID No. 6) and LC+hBactin-L 5′-GGATGCCACAGGACTCCAT-3′ (SEQ ID No. 7) coupled to human Universal Probe Library (UPL) probe number 11 (Roche) resulting in a 124 bp fragment. TRPM2-FL (Exons 20-21) was amplified by RT-PCR from cDNAs using the specific primers: RCH_LONG_TRPM2_U 5′-ACCTCCTCATCGCCATGTT-3′ (SEQ ID No. 8) and RCH_LONG_TRPM2_L 5′-CTTCCAAATCTGGTCCGTGT-3′ (SEQ ID No. 9) coupled to human UPL probe number 11, resulting in a 68 bp fragment. TRPM2-AS (Exons 1-2) was amplified by RT-PCR from cDNAs using the specific primers: RCH-asmel-1F 5′-CCAGGAACCAGAACCAAACT-3′ (SEQ ID No. 10) and RCH-asmel-1R 5′-TGTCCGTCTGCTGAGACATC-3′ (SEQ ID No. 11) coupled to human UPL probe number 57, resulting in a 61 bp fragment. TRPM2-TE was amplified from cDNAs using the primers: UPL-TSS-U 5′-GATGTTTTGGCGGAAGGAC-3′ (SEQ ID No. 12) and UPL-TSS-Rev 5′-CAGGAAGACGTGACGCAAG-3′ (SEQ ID No. 13) coupled to human UPL probe number 6, resulting in a 88 bp intronless fragment. The contribution of genomic DNA contamination to the detected signal was subtracted by running a parallel real-time quantification from a RT-minus reaction, where the RT-enzyme was substituted with water. When RNA was not available to perform the RT-minus control, the contribution of DNA contamination to the detected signal was subtracted by performing a real-time quantification on the same samples, using a couple of primers specific for the Beta-actin promoter: LC-ProBACT-U1 5′-TCTGCAGGAGCGTACAGAAC-3′ (SEQ ID No. 14) and LC-ProBACT-L1 5′-ACATCTCTTGGGCACTGAGC-3′ (SEQ ID No. 15) coupled to human UPL probe number 81 (Roche) resulting in a 85 bp fragment. Real time PCR reactions were performed on a LightCycler 480 instrument (Roche), on 96 multiwell plates (Roche) using LightCycler 480 ProbeMaster Mix (Roche) following the manufacturer instructions. Briefly, all reactions were performed in a final 30 μl volume containing 15 μl of 2× MasterMix, 0.6 μl of each primer (20 μmol/L), 0.3 μl of the corresponding UPL probe (10 μmol/L), and 10 μl of template cDNA. Only for TRPM2-AS the reaction was supplemented with 6 μl of 5× Solution-Q (Qiagen) and the cDNA template lowered to 7 μl. The resulting mix was pre-incubated for 10 minutes at 95° C., then amplification reactions were performed by 45 repetitive cycles of denaturing for 10 seconds at 95° C., annealing for 15 seconds at 58° C. and a final extension plus single acquisition step at 72° C. for 1 second. Levels of target gene transcripts were normalized to transcript levels of a reference gene (Beta-actin) and calculated using a relative quantification model with efficiency correction. Amplification efficiency of primer pairs was calculated from serial dilutions of a representative cDNA template over a concentration range of 3 log orders.

siRNA Experiments.

Two siRNAs designed to target the TRPM2-AS transcript (TRPM2-AS_{—}657 and TRPM2-AS_{—}734), one siRNA designed to target the TRPM2-TE transcript (TRPM2-TE_{—}55) and a control (non-specific) siRNA were synthesized by MWG biotech (Germany) as follows:

1) TRPM2-AS_{—}657, 5′-CCAGUAACUCCGCCCAAAU(dTdT)-3′, (SEQ ID No. 16) target sequence starting at nt 657 of TRPM2-AS.
2) TRPM2-AS_{—}734, 5′-CCACUUACUCAUCCAAGAA(dTdT)-3′, (SEQ ID No. 17) target sequence starting at nt 734 of TRPM2-AS.
3) TRPM2-TE_{—}55, 5′-GAAGGACCACAGAGGAAGU(dTdT)-3′, (SEQ ID No. 18) target sequence starting at nt 55 of TRPM2-TE.
4) Non-specific, 5′-AGGUAGUGUAAUCGCCUUG(dTdT)-3′, (SEQ ID No. 19) no known target in the human transcriptome.

All siRNA sequences were subjected to BLAST search to confirm the absence of similarity to any known additional transcript in the human genome. siRNAs were transfected in the human melanoma cell line Mel Im using the transfection reagent Lipofectamine RNAiMax from Invitrogen. Final siRNA concentration in the medium was 10 nM. Cell were harvested for RNA extraction at 24 hours. qPCR were performed to measure the relative amount of TRPM2-AS, TRPM2-TE and TRPM2-FL transcripts.

### Apoptosis Detection.

Apoptosis was measured after a 72 h transfection with any of the following siRNA: TRPM2-AS_{—}657, TRPM2-AS_{—}734, TRPM2-TE_{—}55 or the non-specific siRNA. Early and late apoptotic cells were assessed by flow cytometry using the Annexin V-Fitc Apoptosis Detection kit (Immunostep, Salamanca, Spain), following manufacturer's instructions. Data acquisition and analysis were done in a FACSort Cytometer (Becton Dickinson, Franklin Lakes, N.J.) using CellQuest and FCS Express software.

### De-novoDNA Methylation of the CpG Island Located Between TRPM2-AS and TRPM2-TE Transcripts.

Three phosphorothioate oligonucleotides were designed in which the cytosines in CpG dinucleotides were replaced by m⁵ CpG. These oligonucleotides are all from the CpG island within the TRPM2 gene (genomic coordinates: chromosome 21:44669421-44670121, NCBI build 35) and were meant to induce methylation within this targeted region using the metod developed by Yao et al. [34].

1) Methylated oligonucleotide 1 (MO1): ^{m}CGG^{m}CGGGGA^{m}CGCTGCCTGAGCTCC^{m}CG (SEQ ID No. 20) at genomic coordinates chr 21: 44669769-44669794, NCBI build 35).
2) Methylated oligonucleotide 2 (MO2): CCCT^{m}CGTAAC^{m}CGCACTG^{m}CGAGTTC (SEQ ID No. 21) at genomic coordinates chr21: 44669697-44669720, NCBI build 35).
3) Methylated oligonucleotide 3 (M03): TGC^{m}CGGGCTGCTGAGTTT^{m}CGC^{m}CGGC (SEQ ID No. 22) at genomic coordinates chr21: 44669657-44669678, NCBI build 35).

Two control oligonucleotides were designed as well:

4) S01 is identical at MO1 except that it doesn't contain m⁵C residues as MO1.
5) MOS is random, i.e., it is not significantly related to the any sequence in the human genome, but contains 3 m⁵ CpG residues and has a base composition similar to MO1, MO2 and MO3: TCCT^{m}CGGGCTGCTGAGTTT^{m}CGC^{m}CGGCCC (SEQ ID No. 23).

The oligonucleotides were purchased from MWG (Germany). The oligonucleotides were purified by high-performance liquid chromatography yielding a purity of more than 95% of full-length oligonucleotides.

Tumor cells were seeded in six-well plates at a density of 2×10⁵ cells/ml and were treated with the following liposome-encapsulated oligonucleotide mixes at a final concentration of 3 μM:
A) MO1+MO2+MO3 (mixture of the three oligonucleotides with each oligonucleotide having a final 1 μM concentration).
B) SO1
C) MOS

Cells were harvested after 24 hours of transfection. DNA was extracted and the methylation status of the CpG island was checked after 24 hours as previously described.

### Statistical Analysis.

Results are expressed as mean±SD (range) or percent. Comparison between groups was made using the Student's unpaired t-test. A p value <0.05 was considered statistically significant. All calculations were performed using the GraphPad Prism software (GraphPad software Inc, San Diego, USA). For real time PCR, data analysis was performed using the relative expression software tool (REST 2005 BETA V1.9.12) [38]. *, p<0.05; **, p<0.01.

### Results

### In Silico Identification of Novel Sense-Antisense Transcription at the TRPM2 Locus.

Using a newly developed version of our AntiHunter software, the authors performed a genome-wide search for antisense transcripts expressed in human ESTs (Expressed Sequence Tags) from melanoma. Several antisense transcripts scattered throughout the genome were identified (data not shown) and here the authors describe one of them, named TRPM2-AS, mapped on chromosome 21. TRPM2-AS is antisense with respect to TRPM2, an ion channel capable of conferring cell-death upon oxidative stress. AntiHunter identified two melanoma antisense ESTs, BF689755 and BF690298, located in the body of the TRPM2 gene and both corresponding to TRPM2-AS. The TRPM2 locus and the relevant molecules herein described are schematically represented in FIG. 1 (numbering refers to the NCBI Build 36.1 (March 2006)). In order to confirm that the transcription start site and the 3′-end of the ESTs were accurate, the authors performed 5′- and 3′-rapid amplification of cDNA ends (RACE) using a commercially available cDNA from malignant melanoma. As a result, it was possible to extend the 5′ end of TRPM2-AS by 292 bases, whereas the 3′ end matched closely that of EST BF689755. The genomic span of the extended TRPM2-AS 875 nucleotides transcript (Genbank accession number: EU362988, SEQ ID No. 1) was 10,974 bp, ranging from position 44,658,901 to position 44,669,874:

| | |
|---|---|
| 1 | CTCCACCTCC CCGAGCCCAA ATGGCCATGC AGGTCGAACA CCAGCTCTGA AGTGGGCAGG |
| 61 | GCCCCCAGGG AGGACAGCCA CGGGAGCTCA GGCAGCGTCC CCGCCGGCAC CTGCCTCTTT |
| 121 | GTGCAGCGGC AGTCAGGGCC CTCGGCAATG AGCTGAACTC GCAGTGCGGT TACGAGGGCA |
| 181 | AATATGCTCC TTGAGGGCCG GCGAAACTCA GCAGCCCGAG GAAGGCTACT GATGTGCATT |
| 241 | TCATAGCCGG CTGCGAATTT AGGAAAACAG ACTTTGCTTC TCGTCACTCA GCCTACGTGA |
| 301 | CCAGGTTCAG ACACAGTCTG CAGCCGCCCG CCTCGCACCC CCACTCTGCA GTGCGGTATG |
| 361 | TGGGGAGCTC AGGGCACAGC AGGCCAGGCC TCCCCGTGGA CGTCCAGGAA CCAGAACCAA |
| 421 | ACTGCCCAGG GCCCCAGAGG GGAAGATGTC TCAGCAGACG GACAGCCGAG GCTCACATGG |
| 481 | CAAGCTCTGG CAGCCTGTCG GTCCCAGGAG AGAGGGGGAG ATGGCAGACG GGAAAAGAAG |
| 541 | CCACCTGCTG GGATGCTGAG ACTCGCTTGC AGGAGCTTTT GGAACTGGCT GAGGTCACAG |
| 601 | CTGGAACCAC TGTGGCCAGC TGGAGTCTGC ACAGCCCGAG TTTCCACCCC AGGGTCCCAG |
| 661 | TAACTCCGCC CAAATGTGCA CACGAGACCT ATGAGGAGAC ATAACTTTCC AGAACCCCCT |
| 721 | TTTCTTCCAC CAGCCACTTA CTCATCCAAG AACCCACCCC CGAACCTTCC CTAATAGAAA |
| 781 | CACTGCATTA AAGCCAGCGC GGGGAGACAG ACGTGAACTG CGCCCCTGTC TCCTTGTGGG |
| 841 | TTGGCCTAGA ATAAAAGCTT TTCTTTTCTC AAAAA |

A 701 bp CpG island, partially superimposed to the TRPM2-AS transcript, was mapped at coordinates 44,669,421-44,670,121 by the “CpGplot” program from the EMBOSS package (www.emboss.org). A prediction for a 570 bp DNA segment containing a PolII promoter was made within the CpG island (coordinates 44,669,484-44,670,053) by “First-EF”, a first-exon and promoter prediction program for human DNA [22], suggesting the CpG island could be involved in the transcriptional regulation of TRPM2-AS (FIG. 1). Intriguingly, when visualizing the locus in a genomic browser, five ESTs from prostate carcinoma, BQ958319, BQ887469, BU543741, BM046691 and BQ920435, appeared to be possibly transcribed from the same CpG island/PolII promoter, (only a representative one, BC041570, is shown for this group of ESTs in the upper part of FIG. 1). Their transcription start site appeared to be located within intron 24 of the TRPM2 gene. In order to precisely map the 5′- and the 3′-ends of this transcript (named Tumor Enriched TRPM2, herein TRPM2-TE), the authors performed another RACE experiment using cDNA from malignant melanoma. The 5′-end of TRPM2-TE was shown to expand by 22 bases with respect to EST BQ958319, whereas the 3′-end was mapped in canonical position with respect to TRPM2 full-length transcript (herein TRPM2-FL). As a result, TRPM2-TE mRNA transcript was determined to be 2,138 nucleotides long (Genbank accession number: EU362987), SEQ ID No. 2:

| | |
|---|---|
| 1 | GACCTGCATG GCCATTTGGG CTCGGGGAGG TGGAGGAGCC CAGATGTTTT GGCGGAAGGA |
| 61 | CCACAGAGGA AGTCCTTGTC CTGCGGGCGG GCACCTGAGC CCGGGCTCCG CCTTGCGTCA |
| 121 | CGTCTTCCTG ACTGTCCCCA GCCTCCCAGA AGGCCGCGGA GGAGCCGGAT GCTGAGCCGG |
| 181 | GAGGCAGGAA GAAGACGGAG GAGCCGGGCG ACAGCTACCA CGTGAATGCC CGGCACCTCC |
| 241 | TCTACCCCAA CTGCCCTGTC ACGCGCTTCC CCGTGCCCAA CGAGAAGGTG CCCTGGGAGA |
| 301 | GAGGAAGGAC GCGGCCGCCA TGGACCCCAT GGGAGAGAAC CCCATGGGCC GCACAGGACT |
| 361 | GCGTGGGCGC GGGAGCCTCA GCTGCTTCGG ACCCAACCAC ACGCTGTACC CCATGGTCAC |
| 421 | GCGGTGGAGG CGGAACGAGG ATGGAGCCAT CTGCAGGAAG AGCATAAAGA AGATGCTGGA |
| 481 | AGTGCTGGTG GTGAAGCTCC CTCTCTCCGA GCACTGGGCC CTGCCTGGGG GCTCCCGGGA |
| 541 | GCCAGGGGAG ATGCTACCTC GGAAGCTGAA GCGGATCCTC CGGCAGGAGC ACTGGCCGTC |
| 601 | TTTTGAAAAC TTGCTGAAGT GCGGCATGGA GGTGTACAAA GGCTACATGG ATGACCCGAG |
| 661 | GAACACGGAC AATGCCTGGA TCGAGACGGT GGCCGTCAGC GTCCACTTCC AGGACCAGAA |
| 721 | TGACGTGGAG CTGAACAGGC TGAACTCTAA CCTGCACGCC TGCGACTCGG GGGCCTCCAT |
| 781 | CCGATGGCAG GTGGTGGACA GGCGCATCCC ACTCTATGCG AACCACAAGA CCCTCCTCCA |
| 841 | GAAGGCAGCC GCTGAGTTCG GGGCTCACTA CTGACTGTGC CCTCAGGCTG GGCGGCTCCA |
| 901 | GTCCATAGAC GTTCCCCCCA GAAACCAGGG CTTCTCTCTC CTGAGCCTGG CCAGGACTCA |
| 961 | GGCTGTTCCT GGGCCCTGCA CATGATGGGG TTTGGTGGAC CCAGTGCCCC TCACGGCTGC |
| 1021 | CGCAAGTCTG CTGCAGATGA CCTCATGAAC TGGAAGGGGT CAAGGTGACC CGGGAGGAGA |
| 1081 | GCTCAAGACA GGGCACAGGC TACTCAGAGC TGAGGGGCCC CTGGGACCCT TGGCCATCAG |
| 1141 | GCGAGGGGCT GGGCCTGTGC AGCTGGGCCC TTGGCCAGAG TCCACTCCCT TCCTGGCTGT |
| 1201 | GTCACCCCGA GCAGCTCATC CACCATGGAG GTCATTGGCC TGAGGCAAGT TCCCCGGAGA |
| 1261 | GTCGGGGTCC CCTGTGGCCC CCTCAGGCCT ATGTCTGTGA GGAAGGGGCC CTGCCACTCT |
| 1321 | CCCCAAGAGG GCCTCCATGT TTCGAGGTGC CTCAACATGG AGCCTTGCCT GGCCTGGGCT |
| 1381 | AGGGGCACTG TCTGAACTCC TGACTGTCAG GATAAACTCC GTGGGGGTAC AGGAGCCCAG |
| 1441 | ACAAAGCCCA GGCCTGTCAA GAGACGCAGA GGGCCCCTGC CAGGGTTGGC CCCAGGGACC |
| 1501 | CTGGGACGAG GCTGCAGAAG CTCTCCCTCC CTACTCCCTG GGAGCCACGT GCTGGCCATG |
| 1561 | TGGCCAGGGA CGGCATGAGC AGGAGGCGGG GACGTGGGGG CCTTCTGGTT TGGTGTCAAC |
| 1621 | AGCTCACAGG AGCGTGAACC ATGAGGGCCC TCAGGAGGGG AACGTGGTAA AACCCAAGAC |
| 1681 | ATTAAATCTG CCATCTCAGG CCTGGCTGGC TCTTCTGTGC TTTCCACAAA TAAAGTTCCT |
| 1741 | GACACGTCCA GGGCCAGGGG CTGTGTGACG GCTGCCTGAA GTTCTCCTCG ATCCCCCGGT |
| 1801 | GAGCTTCCTG CAGCCTGTGG ATGTCCTGCA GCCCCTCAGC CCTACCCCCA AGTTTCTCCT |
| 1861 | CTGACCCATC AGCTCCCTGT CTTCATTTTC CTAAACCTGG GCTCCAGCAT CGTCCCCAAG |
| 1921 | CCCACCAGGC CAGGATGCAG GCATCCACAT GCCCTCCTCC TTGGCTTCCC CTGCGTGGTG |
| 1981 | GTGCCAATGT GCCCTGGCAC CCCTGCAGAG GCTCCGGATG GAGCCTGGGG CTGCCTGGCC |
| 2041 | ACTGAGCACT GGCCGAGGTG ATGCCCACCC TTCCCTGGAC AGGCCTCTGT CTTCCACCTG |
| 2101 | ACCCAAAGCT CTCTAGCCAC CCCCTTGTCC CCAGGTAT |

The transcript spans a genomic region of 17,553 nucleotides from position 44,669,840 to position 44,687,392. Sequencing of the TRPM2-TE transcript revealed other interesting features. It lacked part of exon 26 and the entire exon 27. Removal of part of exon 26 does not affect the putative protein product, as the first methionine is positioned downstream (see below), whereas the ablation of exon 27 corresponds to the already described TRPM2-AC splice variant [23]. The resulting deletion of 34 amino acids removes part of the Nudix domain and it has been reported to impair the capability of TRPM2-FL to be activated by ADP-ribose (ADPR, [23]). However, it leaves intact the segment showing similarity to the CORE region of the NUDT9 domain, containing all the structure required for ADPRase activity [24]. The predicted TRPM2-TE-AC open reading frame ranged from nucleotide 320 to nucleotide 874, encoding for a 184 amino acid protein, whose predicted molecular size is 21,083 Da. The full length TRPM2-TE isoform without the ΔC deletion encoded for a protein of 218 amino acids, with a predicted molecular mass of 25,012 Da. A schematic representation of TRPM2-TE protein products, compared to TRPM2-FL, is shown in FIG. 2. Interestingly, as shown in FIG. 1, all five ESTs from prostate carcinoma displayed these same structural features, suggesting their possible association to cancer. However, selective amplification of the TRPM2-TE-AC region from several melanoma samples revealed the co-existence of a mixed population of TRPM2-TE molecules, both with and without the AC deletion in the same cell line (data not shown).

### Expression Analysis of TRPM2-AS, TRPM2-TE and TRPM2-FL Transcripts in Melanoma.

The authors analyzed TRPM2-AS and TRPM2-TE expression levels in 12 melanoma cell lines and five tumor tissues, with the cell line MaL being the immortalized counterpart of the fresh tumor #3 (FT3). As a control, the authors used normal melanocytes from two independent healthy donors, NHEM1 and NHEM2. The expression level of these two transcripts was also measured in eight normal tissues: heart, brain, placenta, lung, liver, skeletal muscle, kidney and pancreas. Results are shown in FIG. 3a and FIG. 3b, respectively. Both molecules appeared to be up-regulated in over 80% of the cancer samples analyzed, with little or no detectable expression in melanocytes. The eight normal tissues analyzed displayed mostly low levels of these two transcripts. Interestingly, the level of up-regulation of both TRPM2-AS and TRPM2-TE molecules was similar in several of the tumor samples analyzed, suggesting a common mechanism of transcriptional regulation (see below). It should be noted that the MaL cell line displayed significantly lower levels of both TRPM2-AS and TRPM2-TE transcripts with respect to the corresponding fresh tumor sample, FT3. This indicates that quantification experiments performed in cell lines, especially after a high number of passages, might underestimate the level of up-regulation of these two molecules. The expression level of TRPM2-FL was also measured in the same samples. Results are shown in FIG. 3c. In this case, TRPM2-FL expression was detectable, albeit mostly at low levels, both in melanocytes and in 11 out 12 melanoma cell lines. Strikingly, a much higher TRPM2-FL expression was instead observed in all tumor tissues vs. the melanoma cell lines. This discrepancy could be explained by the fact that fresh tumors are often infiltrated by immune cells, which are known to express fairly high levels of TRPM2-FL [13]. Alternatively, it is possible that immortalized cell lines have somehow selected against the capability to express high levels of TRPM2-FL, possibly owing to the proapoptotic nature of this gene [19-21].

Methylation Status of the Shared CpG Island Correlates with the Expression Levels of TRPM2-AS and TRPM2-TE Transcripts.

The authors tried to ascertain if the degree of methylation of the CpG island shared by TRPM2-AS and TRPM2-TE was correlated with the transcriptional activation of these two molecules in melanoma. For this purpose, seven DNAs from the samples analyzed in FIG. 3 were treated with sodium-bisulphite to convert cytosine residues to uracil in single-stranded DNA, under conditions whereby methylated cytosines remain essentially non-reactive. Primers were designed to PCR-amplify a region at coordinates 74-369 of the predicted CpG island. This region contains 19 CpG dinucleotides, whose methylation status was determined after cloning and sequencing of at least 8 clones. The results are shown in FIG. 4. Obvious differences in the methylation patterns of poorly expressing TRPM2-AS and TRPM2-TE samples, namely NHEM1 and NHEM2, vs. relatively strong-expressing cells lines, i.e., Mel Juso, MR299, DettMel, MSR3 and Mel Im, were detected, suggesting that the methylation status of the CpG island contributes negatively to the expression level of both TRPM2-AS and TRPM2-TE.

### Knock-Out of TRPM2-TE Increases Apoptosis and Necrosis in Melanoma.

To analyze the functional role of TRPM2-TE in melanoma cells, the authors down-regulated its expression in melanoma cell line Mel Im by stable transfection with a TRPM2-TE antisense expression construct. Successful down-regulation of TRPM2-TE in cell clones 1 and 2 (TRPM2-TE-AS 1, TRPM2-TE-AS 2) was verified by quantitative RT-PCR (FIG. 5a) and Western blot analysis (FIG. 5b). As a side effect, the level of TRPM2-FL was reduced by this treatment, as shown in FIG. 5c. However, as expected, the knock-out effect was certainly much more efficient for TRPM2-TE than for the TRPM2-FL isoform. Therefore, the authors went on to analyze the amount of apoptosis in cell clones with down-regulated TRPM2-TE expression as well as in control cells. Without stimulation, TRPM2-TE-AS 1 and TRPM2-TE-AS 2 showed a higher level of apoptotic and necrotic cells compared to mock control cells, as shown in FIG. 5d. Stimulation with H₂O₂ enhanced this effect (data not shown).

### Overexpression of TRPM2-FL Reduces Melanoma Proliferation Rate and Increases its Caspase Activity.

The authors generated melanoma cell clones which expressed higher TRPM2-FL levels than parental cells by stable transfection of Mel Im cells with an expression construct for TRPM2-FL. Over-expression was tested by quantitative RT-PCR revealing that the cell clones 3 and 7 had a strongly increased expression of TRPM2-FL when compared to mock-transfected cell clones mock 1 and mock 2 (FIG. 6a). Cells expressing high levels of TRPM2-FL showed reduced proliferation compared to mock transfected cells and Mel Im cells (FIG. 6b). These results are consistent with the analysis of the amount of apoptotic cells (data not shown). Strikingly, while clone 3 had greater expression than clone 7, there is no difference between these two clones in terms of proliferation rate. This is probably due to the fact that the capability to slow down cell proliferation via FL_TRPM2 has been already saturated by the expression level reached using clone 7. Therefore, a further increase of FL_TRPM2 cellular concentration, such as that obtained using clone 3, doesn't bring any further decrease in the proliferation rate. TRPM2-FL expressing cell clones had higher levels of caspase activity compared to Mel Im cells and mock-transfected cell clones. This effect could be intensified by stimulating cells with H₂O₂ (FIG. 6c). Transient transfection of the TRPM2-FL cell clone 3 with a TRPM2-TE expression plasmid protected cells from apoptosis. This effect was even stronger after stimulating cells with H₂O₂, as shown in FIG. 6d.

### TRPM2-AS and TRPM2-TE are Also Overexpressed in Other Tumor Types.

In order to investigate the possibility that overexpression of TRPM2-AS and TRPM2-TE transcripts is a wider phenomenon in cancer, quantitative RT-PCR experiments were performed on other tumor types. In FIG. 7, four matched neoplastic/non-neoplastic lung tissue (LT) samples, with the normal tissue adjacent to the tumor, namely LT1_N and LT1_T, LT2_N and LT2_T, LT3_N and LT3_T, LT4_N and LT4_T, were analyzed. Results are shown as fold induction with respect to a lung cDNA from an independent healthy donor. Tumor samples displayed higher levels of TRPM2-AS and TRPM2-TE transcripts with respect to their matched normal tissue. Interestingly, LT2 tissue sample, showing the highest TRPM2-TE levels among the cancer samples analyzed, also displayed high levels of this transcript in its healthy counterpart, suggesting the possibility that the spreading of tumor cells had already occurred in the ostensibly healthy tissue.

Expression analysis of TRPM2-AS, TRPM2-TE and TRPM2-FL transcripts in resected non-small cell lung cancer patients revealed that TRPM2-AS was over-expressed in 17 samples (63%), under-expressed in 9 samples (33%) and equally expressed in one sample (4%). TRPM2-TE was over-expressed in 24 samples (89%), under-expressed in 2 samples (7%) and equally expressed in one sample (4%). TRPM2-FL was over-expressed in 9 samples (33%), under-expressed in 13 (48%) samples and equally expressed in 5 samples (18%) (FIG. 8). These results demonstrate that expression levels of TRPM2-AS allow to discriminate between normal lung tissue and lung cancer in 63% of cases (0% false positives). Moreover, expression levels of TRPM2-TE allow to discriminate between normal lung tissue and lung cancer in 89% of cases (0% false positives).

It is conceivable that TRPM2-TE and TRPM2-AS exert their function by counteracting the action of TRPM2-FL within the cell. Therefore, since the overall capability of TRPM2-FL to induce cell death could be affected by the amounts of these two molecules, the authors calculated the following value for each sample from the fold induction data: TRPM2-FL-TRPM2-AS-TRPM2-TE. This means that this new value, named TRPM2-FL Relative Fold Induction (TRFI), is calculated by subtracting the figure of TRPM2-AS and TRPM2-TE fold induction from the TRPM2-FL one. In order to keep in account saturating concentration of these molecules in the cell, the fold induction values used for calculation were kept within the −/+10-fold range. For example, from FIG. 8, the fold induction of TRPM2-AS, TRPM2-TE ad TRPM2-FL in LC1_{—}1A samples is, respectively, the following: −3.76, 6.32 and −3.32. This yields a TRFI of −5.88. Another example: sample LC12_{—}1B has undetectable levels of TRPM2-AS (equated to −10), a fold induction of 292.87 times of TRPM2-AS (equated to +10) and −1.93 times for TRPM2-FL. The resulting TRFI is −1.93. The TRFI was calculated for all the lung cancer samples. In Table I, lung cancer samples from stage I and II are sorted on the base of the overall survival (OS) of respective patients expressed in month.

TABLE I
TRPM2-FL Relative Fold Induction

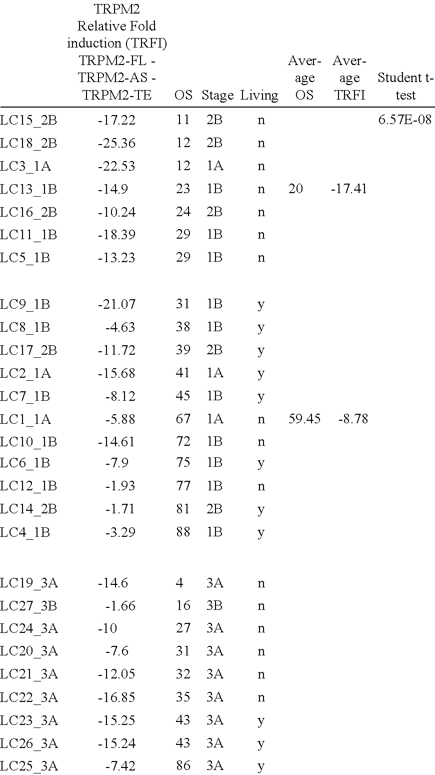

The average TRFI of short term survival patients (0-30 months, dark grey boxes) differs from the one of long term survival patients (31-88 months, light grey boxes): −17.41 vs. −8.77. A t-test applied to the TRFI values of both distributions determined that this difference was significant (p-value: 6.57E-08). No significant difference was found in stage 3 samples (data not shown). Tumor stage (TS), overall surviving (OS) after surgery and whether patient was still living after the end of the clinical follow-up is also shown in the table. This data demonstrate that the TRFI value is a significant prognostic marker in lung cancer. The month survival of patients from stage I and stage II having a TRFI<−10 (dotted line; n=11; average survival: 29 months) and a TRFI>−10 (solid line; n=7; average survival: 67 months) was plotted in a Kaplan-Meyer survival plot in FIG. 9. A log-rank test determined the significance of these different distribution (p-value: 0.001). Quantitative RT-PCR experiments were also performed to quantify TRPM2-AS and TRPM2-TE transcripts in both normal (HB100, MCF-10) and breast cancer cell lines (MDAMB231, MDAMB453, MDAMB468, T47D, BT20, ZR751, MDA435, BT474, MCF7). Results are displayed as arbitrary units in FIG. 10a and FIG. 10b, respectively. Quantification of TRPM2-AS revealed that it was undetectable in the two healthy controls, as well as in MDAMB231, MDAMB453, ZR751 and MCF7 breast cancer cell lines. However, its expression was clearly upregulated in the remaining five breast cancer cell lines (55%), with the highest and lowest values observed in MDA435 and MDAMB468 cell lines. Expression analysis of TRPM2-TE transcript showed that it was undetectable in the healthy MCF-10 cell line as well as in the MDAMB231 breast cancer cell line. It had a moderate expression in both normal HB100 and low-invasiveness MDA-MB-468 cell lines [25] and was significantly upregulated in all of the remaining 7 cell lines (77%). Also, the highest and lowest expression values in the tumor cell lines were found in the MDA435 and MDAMB468 cell lines, respectively.

siRNAs Targeting TRPM2-AS and TRPM2-TE Transcripts Down-Regulate their Expression in Melanoma Cells

siRNAs experiments, shown in FIG. 11, were designed to target the TRPM2-AS transcript or the TRPM2-TE transcript. They showed that, respect to mock-transfected cells, TRPM2-AS_{—}657 siRNA was able to ablate 69% of TRPM2-AS expression, TRPM2-AS_{—}734 was able to knock-out 75% of TRPM2-AS expression, whereas TRPM2-TE_{—}55 was able to remove 56% of TRPM2-TE molecules. Interestingly, these three siRNA had a cross inhibitory effect: TRPM2-AS_{—}657 removed also 84% of TRPM2-TE molecules, TRPM2-AS_{—}734 removed also 70% of TRPM2-TE transcripts, whereas TRPM2-TE 55 remove 30% of TRPM2-AS RNA. No significant down-regulation of TRPM2-AS and TRPM2-TE transcripts was observed when transfecting cells with a non-specific siRNA. These results demonstrate that the above siRNAs can properly abolish TRPM2-AS and TRPM2-TE expression. Such decreased expression induces an increased apoptosis in melanoma cells, as shown in FIG. 13 (see figure legend for details). Therefore inhibition of TRPM2-AS and/or TRPM2-TE expression can be used to inhibit tumour cells. The inhibition can be performed by means of siRNA technology or any other means known in the art.

### De-Novo Methylation of the CpG Island Located Between TRPM2-AS and TRMP2-TE Transcripts

Following the protocol of Yao et al [34], the authors designed three methylated oligonucleotides aimed at inducing new methylation in melanoma cells of the CpG island located in between TRPM2-AS and TRPM2-TE transcripts. Three methylated oligonucleotides, MON1, MON2 and MON3, targeting the CpG island were designed. Two negative control oligonucleotides, SO1 and MOS, were designed as well. The first of these two control oligonucleotides, SO1, had the same sequence of MO1, but it was completely not-methylated. The second control oligo, MOS, was not significantly related to any sequence in the human genome. Oligonucleotides were transfected in melanoma Mel Im cells. After 24 hours, DNA was extracted and treated with sodium bisulphite to convert cytosine residues to uracil in single-stranded DNA, under conditions whereby methylated cytosines remain essentially non-reactive. As previously described, primers were used to PCR-amplify the region at coordinates 74-369 of the predicted CpG island, containing 19 CpG dinucleotides, whose methylation status was determined after cloning and sequencing of 5 clones. The results are shown in FIG. 12, with open (∘) and closed () circles indicating, respectively, non-methylated and methylated CpG sites. As a result, the mix of the three methylated oligonucleotides partially methylated 3 out of 5 clones, whereas non-methylation was induced both by the SO1 and the MOS control oligonucleotides.

### DISCUSSION

In the study presented here, the authors mined the EST melanoma database with their recently developed software AntiHunter and identified a new antisense transcript, TRPM2-AS. Natural Antisense Transcripts (NATs) are supposed to negatively regulate the conjugated sense transcript by means of a variety of regulatory mechanisms [9]. The sense partner of TRPM2-AS is TRPM2, a gene whose protein product encodes for an ion channel capable of conferring cell-death upon oxidative stress. Although the direct involvement of TRPM2 in tumor cell death has been shown only very recently [21], the authors contemplated that activation of TRPM2-AS could interfere with the proapoptotic role of TRPM2 during the process of cancerogenesis.

Quantitative RT-PCR experiments revealed transcriptional activation of TRPM2-AS in about 80% of melanoma cell lines and tumor tissues vs. controls. Visualization of the AntiHunter output, using a genome browser, allowed the authors to hint at other interesting features of the investigated region. First, a CpG island/Pol II promoter, mapped within intron 24 of TRPM2, appeared to be closely positioned to the TRPM2-AS transcription start site. Second, five ESTs from prostate carcinoma appeared to have a transcription start site located within the same intron 24, suggesting the CpG island could serve also as a promoter for a new Tumor Enriched TRPM2 transcript (TRPM2-TE). Quantitative RT-PCR experiments showed that TRPM2-TE expression was activated in melanoma and coupled to the expression of TRPM2-AS suggesting a bidirectional role for the shared promoter region. Sequencing of bisulphite treated DNAs of several melanoma samples indicated the methylation status of the CpG island was at least partially correlated with the expression levels of TRPM2-AS and TRPM2-TE transcripts. Since this CpG island appeared to be mostly hypermethylated in normal melanocytes, it is conceivable that its demethylation could activate TRPM2-AS and TRPM2-TE expression in melanoma, contributing to the functional downregulation of TRPM2-FL.

No obvious indication on the regulatory role of TRPM2-AS over FL-TRPM2 could be determined by the qPCR data shown in FIG. 3a and FIG. 3c. However, as TRPM2-FL activity depends also on the strength of its own promoter, the effect of TRPM2-AS expression on its transcription could be not easily detected by this experiment. Several mechanisms involved in the regulation of a sense transcript by its antisense partner have already been described (reviewed in [9, 26-30]): 1) transcriptional interference; 2) RNA masking; 3) double-stranded RNA (dsRNA)-dependent mechanisms and 4) antisense RNA-mediated CpG island methylation. Intriguingly, a recent paper describes the epigenetic silencing of the tumor suppressor gene p15 by its antisense RNA through heterochromatin formation, but not DNA methylation [31]. In the present case, direct sequencing of several TRPM2-FL cDNAs failed to detect any deamination (A to I conversion) in the region encompassing exons 21-24, i.e., those overlapped by the primary TRPM2-AS transcript (data not shown), which suggests that RNA editing is unlikely to occur. Since primer extension experiments mapped the TRPM2-AS 3′-end in the body of the TRPM2-FL gene, about 60 Kb apart from its 5′-end, then antisense RNA-mediated CpG island methylation can be excluded.

Experimental evidence for the dominant-negative role of TRPM2-TE was determined by the present invention experiments. Knock-down of this transcript significantly increased the susceptibility of melanoma cells to apoptosis and necrosis. Since the present experimental approach to TRPM2-TE depletion also removed a significant fraction of TRPM2-FL from the cells (see FIG. 5b), the dominant negative role of TRPM2-TE could have been underestimated by the present experiments. According to the above functional data, overexpression of TRPM2-FL in melanoma cell clones interfered with some traits of the tumoral phenotype leading to an increased doubling time in proliferation assays as well as higher caspase activity. Along the same lines, transient overexpression of TRPM2-TE in the TRPM2-FL over-expressing cell clones protected these cells from apoptosis, suggesting that restoration of TRPM2-FL activity in melanoma cells could contribute to shift them toward a less aggressive state and/or apoptosis. Thus, TRPM2-TE inhibits the function of TRPM2-FL. Other truncated iso forms of TRPM2-FL have already been shown to work as dominant negative [17,32]. Alternatively, or in adjunct, since TRPM2-TE maintains the CORE portion of the Nudix domain required for ADPRase activity (see FIG. 3), it might simply work by sequestering/consuming the cellular supply of ADPR needed for TRPM2 activation. RNAi experiment presented in this invention further demonstrated the anti-apoptotic role of TRPM2TE molecule. They also indicated the anti-apoptotic role of TRPM2-AS transcript.

Quantitative RT-PCR experiments performed in lung and breast cancer (FIG. 7 and FIG. 10), seem to support the idea that activation of TRPM2-AS and TRPM2-TE transcripts is a phenomenon common to other tumors.

A large sequencing effort directed toward human colorectal and breast cancer DNA gene coding regions and splicing consensi failed to find any mutation within TRPM2-FL [2]. Therefore, it is conceivable that TRPM2-FL inactivation in cancer is mainly obtained by epigenetic means, such as those described in the present invention. A possible explanation is that TRPM2-FL function could be needed in the initial phases of tumor development, while becoming harmful at later stages. Accordingly, since metastasis is a cellular heterogeneous process, it is unlikely to be mediated by permanent genetic mutations. However, epigenetically mediated gene silencing is an excellent candidate for supporting such cellular dynamics [33]. The kind of epigenetic regulation is shown here to allow the fine tuning of the TRPM2-FL requirements of the tumor by acting on the methylation status of the inner CpG island. Another important player in this scenario should be the promoter of the TRPM2-FL gene, whose activation strength should also contribute to the functional activity of TRPM2-FL.

From the data reported in the present invention, restoration of TRPM2-FL activity in cancer cells is an attractive therapeutic opportunity. Since inactivation of TRPM2-FL is likely to correlate with the hypomethylation of the CpG island shared by TRPM2-AS and TRPM2-TE transcripts, a simple way to restore its activity is to artificially methylate it de novo. Experimental evidence indicates the feasibility of this approach, obtained by delivering a methylated oligonucleotide complementary to the targeted CpG island to the nucleus [34]. The authors delivered a methylated oligonucleotide to the shared CpG island with positive results, as shown in FIG. 12.

The systematic identification of antisense transcripts in the EST database holds great promise for the identification of new and interesting biological and pathological phenomena. With the advent of new sequencing technologies that generate order-of-magnitudes larger sequencing data at fractional costs [35], it is conceivable that the identification of regulatory antisense transcripts obtained by mining the EST database will gain even more ground in the future.

### REFERENCES

1. Vogelstein B, Kinzler K W. Nat Med. 2004; 10:789-799.
2. Sjoblom T, Jones S, Wood L D, et al Science. 2006; 314:268-274.
3. Wood L D, Parsons D W, Jones S, et al. Science. 2007; 318:1108-1113.
4. Baylin S B, Ohm J E. Nat Rev Cancer 2006; 6:107-116.
5. Feinberg A P, Ohlsson R, Henikoff S. Nat Rev Genet. 2006; 7:21-23.
6. Ting A H, McGarvey K M, Baylin S B. Genes Dev. 2006; 20:3215-3231.
7. Baylin S B, Herman J G. In: Ehrlich M, ed. DNA. MA, USA: Eaton publishing, 2000:293-309.
8. Ehrlich M. In: Ehrlich M, eds. DNA Alterations in Cancer. MA, USA: Eaton publishing, 2000:273-291.
9. Lavorgna G, et al., Trends Biochem Sci. 2004; 29:88-94.
10. Lavorgna G, et al., Nucleic Acids Res. 2005; 33:W665-668.
11. Nagamine K, Kudoh J, Minoshima S, et al. Genomics. 1998; 54:124-131.
12. Clapham D E. Nature. 2003; 426:517-524.
13. Sano Y, Inamura K, Miyake A, et al. Science. 2001; 293:1327-1330.
14. Kuhn F J, Heiner I, Luckhoff A. Pflugers Arch. 2005; 451:212-219.
15. Perraud A L, Takanishi C L, Shen B, et al. J Biol Chem. 2005; 280:6138-6148.
16. Hara Y, Wakamori M, Ishii M, et al. Mol Cell. 2002; 9:163-173.
17. Zhang W, Chu X, Tong Q, et al. J Biol Chem. 2003; 278:16222-16229.
18. Miller B A. J Membr Biol. 2006; 209:31-41.
19. Yang K T, Chang W L, Yang P C, et al. Cell Death Differ. 2006; 13:1815-1826.
20. Zhang W, et al. Am J Physiol Cell Physiol. 2006; 290:C1146-1159.
21. Ishii M, Oyama A, Hagiwara T, et al. Anticancer Res. 2007; 27:3987-92.
22. Davuluri R V, Grosse I, Zhang M Q. Nat Genet. 2001; 29:412-417.
23. Wehage E, et al., J Biol Chem. 2002; 277:23150-23156.
24. Perraud A L, Shen B, Dunn C A, et al. J Biol Chem. 2003; 278:1794-801.
25. Thompson E W, Paik S, Brünner N, et al. J Cell Physiol. 1992; 150:534-544.
26. Munroe S H. J Cell Biochem. 2004; 93:664-671.
27. Werner A, Berdal A. Physiol Genomics. 2005; 23(2):125-131.
28. Lapidot M, Pilpel Y. EMBO Rep. 2006; 7:1216-1222.
29. Tufarelli C. Philos Trans R Soc Lond B Biol Sci. 2006; 361:67-79.
30. Mazo A, et al., J Cell Sci. 2007; 120:2755-2761.
31. Yu W, Gius D, Onyango P, et al. Nature 2008; 451:202-206.
32. Uemura T, et al., Biochem Biophys Res Commun. 2005; 328:1232-1243.
33. Jones P A, Baylin S B. Nat Rev Genet. 2002; 3:415-428.
34. Yao X, Hu J F, Daniels M, et al. J Clin Invest. 2003; 111:265-273.
35. Ryan D, et al., Trends Biotechnol. 2007; 25:385-389.
36. Jacob K, Wach F, Holzapfel U, et al. Melanoma Res. 1998; 8:211-219.
37. Poser I, Tatzel J, Kuphal S, Bosserhoff A K. Oncogene. 2004; 23:6115-24.
38. Pfaffl M W, Horgan G W, Dempfle L. Nucleic Acids Res. 2002; 30:e36.

1. A nucleic acid molecule being able to modulate the expression of the transient receptor potential cation channel, subfamily M, member 2 (TRPM2) gene and to modulate apoptosis.

2. The nucleic acid molecule according to claim 1 being antisense in respect to the TRPM2 gene.

3. The nucleic acid molecule according to claim 2 consisting of SEQ ID No. 1.

4. The nucleic acid molecule according to claim 1 being sense in respect to the TRPM2 gene.

5. The nucleic acid molecule according to claim 4 consisting of SEQ ID No. 2.

6. An expression vector and/or a viral transformation (transduction) system and/or a transformation system based on ‘gene gun’ technology comprising the nucleic acid molecule according to claim 1.

7. A host cell transformed with the expression vector according to claim 6.

8. A non human transgenic animal bearing the nucleic acid molecule according to claim 1.

9. The nucleic acid molecule according to claim 1 for medical use.

10. The nucleic acid molecule according to claim 1 for use as anti-neurodegenerative disease.

11. The nucleic acid molecule according to claim 10 wherein the neurodegenerative disease is selected from the group of Alzheimer's or Parkinson's disease.

12. The nucleic acid molecule according to claim 1 for use as anti-apoptotic agent in ischemic cells.

13. The nucleic acid molecule according to claim 11 wherein the ischemic cells are selected from the group of: neurons, cardiomyocites, kidney cells, lung cells, pancreas beta-cells.

14. The nucleic acid molecule according to claim 1 for use as CpG island methylation agent.

15. The nucleic acid molecule according to claim 14 belonging to the group of methylated oligonucleotides MO1, MO2 and MO3, for use as de-novo methylation agents of the CpG island located between TRPM2-AS and TRPM2-TE transcripts.

16. The nucleic acid molecule according to claim 1 for the diagnosis and prognosis of cancer.

17. (canceled)

18. A molecule able to down regulate the nucleic acid molecule according to claim 1 for use as a pro-apoptotic and/or pro-necrosis therapy agent in cancer cells.

19. A method to down-regulate TRPM2-TE and/or TRPM2-AS transcripts in melanoma cells characterized by methylating the CpG island located between TRPM2-AS and TRPM2-TE transcripts.

20. A method to induce apoptosis in melanoma cells characterized by down regulating TRPM2-TE and/or TRPM2-AS transcripts in said melanoma cells.

21. The method of claim 20 comprising stimulating the cells with H₂O₂.

22. A prognostic index, named TFRI, calculated from the fold-induction figures of TRPM2-FL, TRPM2-TE and TRPM2-AS transcripts in cancer vs. healthy lung, capable to predict the short- or long-term survival of stage I and stage II lung cancer patients.


SEQUENCE LISTING (23 sequences)

SEQ ID NO: 1 - DNA, 875 bp, Artificial Sequence
  synthetic TRPM2-AS transcript
ctccacctccccgagcccaaatggccatgcaggtcgaacaccagctctgaagtgggcagggcccccagggaggacagccacgggagctcaggcagcgtccccgccggcacctgcctctttgtgcagcggcagtcagggccctcggcaatgagctgaactcgcagtgcggttacgagggcaaatatgctccttgagggccggcgaaactcagcagcccgaggaaggctactgatgtgcatttcatagccggctgcgaatttaggaaaacagactttgcttctcgtcactcagcctacgtgaccaggttcagacacagtctgcagccgcccgcctcgcacccccactctgcagtgcggtatgtggggagctcagggcacagcaggccaggcctccccgtggacgtccaggaaccagaaccaaactgcccagggccccagaggggaagatgtctcagcagacggacagccgaggctcacatggcaagctctggcagcctgtcggtcccaggagagagggggagatggcagacgggaaaagaagccacctgctgggatgctgagactcgcttgcaggagcttttggaactggctgaggtcacagctggaaccactgtggccagctggagtctgcacagcccgagtttccaccccagggtcccagtaactccgcccaaatgtgcacacgagacctatgaggagacataactttccagaacccccttttcttccaccagccacttactcatccaagaacccacccccgaaccttccctaatagaaacactgcattaaagccagcgcggggagacagacgtgaactgcgcccctgtctccttgtgggttggcctagaataaaagcttttcttttctcaaaaa

SEQ ID NO: 2 - DNA, 2138 bp, Artificial Sequence
  synthetic TRPM2-TE transcript
gacctgcatggccatttgggctcggggaggtggaggagcccagatgttttggcggaaggaccacagaggaagtccttgtcctgcgggcgggcacctgagcccgggctccgccttgcgtcacgtcttcctgactgtccccagcctcccagaaggccgcggaggagccggatgctgagccgggaggcaggaagaagacggaggagccgggcgacagctaccacgtgaatgcccggcacctcctctaccccaactgccctgtcacgcgcttccccgtgcccaacgagaaggtgccctgggagagaggaaggacgcggccgccatggaccccatgggagagaaccccatgggccgcacaggactgcgtgggcgcgggagcctcagctgcttcggacccaaccacacgctgtaccccatggtcacgcggtggaggcggaacgaggatggagccatctgcaggaagagcataaagaagatgctggaagtgctggtggtgaagctccctctctccgagcactgggccctgcctgggggctcccgggagccaggggagatgctacctcggaagctgaagcggatcctccggcaggagcactggccgtcttttgaaaacttgctgaagtgcggcatggaggtgtacaaaggctacatggatgacccgaggaacacggacaatgcctggatcgagacggtggccgtcagcgtccacttccaggaccagaatgacgtggagctgaacaggctgaactctaacctgcacgcctgcgactcgggggcctccatccgatggcaggtggtggacaggcgcatcccactctatgcgaaccacaagaccctcctccagaaggcagccgctgagttcggggctcactactgactgtgccctcaggctgggcggctccagtccatagacgttccccccagaaaccagggcttctctctcctgagcctggccaggactcaggctgttcctgggccctgcacatgatggggtttggtggacccagtgcccctcacggctgccgcaagtctgctgcagatgacctcatgaactggaaggggtcaaggtgacccgggaggagagctcaagacagggcacaggctactcagagctgaggggcccctgggacccttggccatcaggcgaggggctgggcctgtgcagctgggcccttggccagagtccactcccttcctggctgtgtcaccccgagcagctcatccaccatggaggtcattggcctgaggcaagttccccggagagtcggggtcccctgtggccccctcaggcctatgtctgtgaggaaggggccctgccactctccccaagagggcctccatgtttcgaggtgcctcaacatggagccttgcctggcctgggctaggggcactgtctgaactcctgactgtcaggataaactccgtgggggtacaggagcccagacaaagcccaggcctgtcaagagacgcagagggcccctgccagggttggccccagggaccctgggacgaggctgcagaagctctccctccctactccctgggagccacgtgctggccatgtggccagggacggcatgagcaggaggcggggacgtgggggccttctggtttggtgtcaacagctcacaggagcgtgaaccatgagggccctcaggaggggaacgtggtaaaacccaagacattaaatctgccatctcaggcctggctggctcttctgtgctttccacaaataaagttcctgacacgtccagggccaggggctgtgtgacggctgcctgaagttctcctcgatcccccggtgagcttcctgcagcctgtggatgtcctgcagcccctcagccctacccccaagtttctcctctgacccatcagctccctgtcttcattttcctaaacctgggctccagcatcgtccccaagcccaccaggccaggatgcaggcatccacatgccctcctccttggcttcccctgcgtggtggtgccaatgtgccctggcacccctgcagaggctccggatggagcctggggctgcctggccactgagcactggccgaggtgatgcccacccttccctggacaggcctctgtcttccacctgacccaaagctctctagccacccccttgtccccaggtat

SEQ ID NO: 3 - DNA, 22 bp, Artificial Sequence
  synthetic primer
ttgatttgatttggtttttgga

SEQ ID NO: 4 - DNA, 22 bp, Artificial Sequence
  synthetic primer
caaaacaaaaacttcctctata

SEQ ID NO: 5 - DNA, 25 bp, Artificial Sequence
  synthetic primer
tgtagattgtgtttgaatttggtta

SEQ ID NO: 6 - DNA, 19 bp, Artificial Sequence
  synthetic primer
tcctcctggagaagagcta

SEQ ID NO: 7 - DNA, 19 bp, Artificial Sequence
  synthetic primer
ggatgccacaggactccat

SEQ ID NO: 8 - DNA, 19 bp, Artificial Sequence
  synthetic primer
acctcctcatcgccatgtt

SEQ ID NO: 9 - DNA, 20 bp, Artificial Sequence
  synthetic primer
cttccaaatctggtccgtgt

SEQ ID NO: 10 - DNA, 20 bp, Artificial Sequence
  synthetic primer
ccaggaaccagaaccaaact

SEQ ID NO: 11 - DNA, 20 bp, Artificial Sequence
  synthetic primer
tgtccgtctgctgagacatc

SEQ ID NO: 12 - DNA, 19 bp, Artificial Sequence
  synthetic primer
gatgttttggcggaaggac

SEQ ID NO: 13 - DNA, 19 bp, Artificial Sequence
  synthetic primer
caggaagacgtgacgcaag

SEQ ID NO: 14 - DNA, 20 bp, Artificial Sequence
  synthetic primer
tctgcaggagcgtacagaac

SEQ ID NO: 15 - DNA, 20 bp, Artificial Sequence
  synthetic primer
acatctcttgggcactgagc

SEQ ID NO: 16 - DNA, 21 bp, Artificial Sequence
  synthetic TRPM2-AS 657 siRNA
ccaguaacuccgcccaaautt

SEQ ID NO: 17 - DNA, 21 bp, Artificial Sequence
  synthetic TRPM2-AS 734 siRNA
ccacuuacucauccaagaatt

SEQ ID NO: 18 - DNA, 21 bp, Artificial Sequence
  synthetic TRPM2-TE 55 siRNA
gaaggaccacagaggaagutt

SEQ ID NO: 19 - DNA, 21 bp, Artificial Sequence
  synthetic non specific siRNA
agguaguguaaucgccuugtt

SEQ ID NO: 20 - DNA, 26 bp, Artificial Sequence
  synthetic methylated oligonucleotide MO1
cggcggggacgctgcctgagctcccg

SEQ ID NO: 21 - DNA, 24 bp, Artificial Sequence
  synthetic methylated oligonucleotide MO2
ccctcgtaaccgcactgcgagttc

SEQ ID NO: 22 - DNA, 25 bp, Artificial Sequence
  synthetic methylated oligonucleotide MO3
tgccgggctgctgagtttcgccggc

SEQ ID NO: 23 - DNA, 28 bp, Artificial Sequence
  synthetic methylated oligonucleotide MOS
tcctcgggctgctgagtttcgccggccc